SEQUENCE LISTING (21 sequences)

SEQ ID NO: 1 - protein, 8 aa, Artificial Sequence
  Synthetic Polypeptide
DYKDDDDK

SEQ ID NO: 2 - protein, 242 aa, Artificial Sequence
  Synthetic Polypeptide
QVQLQQSGPGLVKPSQTLSLTCAISGDSVSSNSAAWNWIRQSPSRGLEWLGRTYYRSKWYNDYAVSVKSRITIDPDTSKNQFSLQLNSVTPEDTAMYYCAREGDDAFDIWGQGTMVTVSSGGGGSGSGASSGGGSSYVLTQLPSVSVSPGQTARITCGGNNIGSKSVHWYQQKPGQAPVLVVYDDSDRPSGIPERFSGSNSGNTATLTISGTQAMDEADYYCQAWDSGTAVFGGGTKVTVLG

SEQ ID NO: 3 - protein, 120 aa, Artificial Sequence
  Synthetic Polypeptide
QVQLQQSGPGLVKPSQTLSLTCAISGDSVSSNSAAWNWIRQSPSRGLEWLGRTYYRSKWYNDYAVSVKSRITIDPDTSKNQFSLQLNSVTPEDTAMYYCAREGDDAFDIWGQGTMVTVSS

SEQ ID NO: 4 - protein, 10 aa, Artificial Sequence
  Synthetic Polypeptide
GDSVSSNSAA

SEQ ID NO: 5 - protein, 9 aa, Artificial Sequence
  Synthetic Polypeptide
TYYRSKWYN

SEQ ID NO: 6 - protein, 9 aa, Artificial Sequence
  Synthetic Polypeptide
AREGDDAFD

SEQ ID NO: 7 - protein, 107 aa, Artificial Sequence
  Synthetic Polypeptide
SYVLTQLPSVSVSPGQTARITCGGNNIGSKSVHWYQQKPGQAPVLVVYDDSDRPSGIPERFSGSNSGNTATLTISGTQAMDEADYYCQAWDSGTAVFGGGTKVTVLG

SEQ ID NO: 8 - protein, 6 aa, Artificial Sequence
  Synthetic Polypeptide
NIGSKS

SEQ ID NO: 9 - protein, 3 aa, Artificial Sequence
  Synthetic Polypeptide
DDS

SEQ ID NO: 10 - protein, 9 aa, Artificial Sequence
  Synthetic Polypeptide
QAWDSGTAV

SEQ ID NO: 11 - DNA, 726 bp, Artificial Sequence
  Synthetic Polynucleotide
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcgacccagacacatccaagaaccagttctccctgcagctgaactctgtgactcccgaggatacggctatgtattactgtgcaagagagggagatgatgcttttgatatctggggccaagggacaatggtcaccgtctcctcaggaggtggcgggtctggtagtggcgctagcagtggtggcggatcctcctatgtgctgactcagctaccctcggtgtcagtgtccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacagccactctgaccatcagcgggacccaggctatggatgaggctgactattactgtcaggcgtgggacagcggcactgcggtattcggcggagggaccaaggtcaccgtcctaggt

SEQ ID NO: 12 - protein, 247 aa, Artificial Sequence
  Synthetic Polypeptide
QVQLQQSGPGLVKPSQTLSLTCAISGDSVSSYNAAWTWIRQSPSRGLEWLGRTYYGSKWYTDYAVSVKSRITINPDTSKNQFSLQLNSVTPEDTAVYYCARESELRADAFDIWGQGTTVTVSSGGGGSGSGASGGGGSSSELTQDPAVSVALGQTVRITCQGDSLRSYYASWYQQKPGQAPVLVIYGKNNRPSGIPDRFSGSSSGTTASLTITGAQAEDEADYYCNSRDSSGNHLVFGGGTKVTVLG

SEQ ID NO: 13 - protein, 123 aa, Artificial Sequence
  Synthetic Polypeptide
QVQLQQSGPGLVKPSQTLSLTCAISGDSVSSYNAAWTWIRQSPSRGLEWLGRTYYGSKWYTDYAVSVKSRITINPDTSKNQFSLQLNSVTPEDTAVYYCARESELRADAFDIWGQGTTVTVSS

SEQ ID NO: 14 - protein, 10 aa, Artificial Sequence
  Synthetic Polypeptide
GDSVSSYNAA

SEQ ID NO: 15 - protein, 9 aa, Artificial Sequence
  Synthetic Polypeptide
TYYGSKWYT

SEQ ID NO: 16 - protein, 13 aa, Artificial Sequence
  Synthetic Polypeptide
ARESELRADAFDI

SEQ ID NO: 17 - protein, 109 aa, Artificial Sequence
  Synthetic Polypeptide
SSELTQDPAVSVALGQTVRITCQGDSLRSYYASWYQQKPGQAPVLVIYGKNNRPSGIPDRFSGSSSGTTASLTITGAQAEDEADYYCNSRDSSGNHLVFGGGTKVTVLG

SEQ ID NO: 18 - protein, 6 aa, Artificial Sequence
  Synthetic Polypeptide
SLRSYY

SEQ ID NO: 19 - protein, 3 aa, Artificial Sequence
  Synthetic Polypeptide
GKN

SEQ ID NO: 20 - protein, 11 aa, Artificial Sequence
  Synthetic Polypeptide
NSRDSSGNHLV

SEQ ID NO: 21 - DNA, 741 bp, Artificial Sequence
  Synthetic Polynucleotide
caggtacagctgcagcagtcaggtccaggactggtgaagccctcgcagaccctctcactcacctgtgccatctccggggacagtgtctctagctacaatgctgcttggacctggatcaggcagtccccatcgagaggccttgagtggctgggaaggacatactacgggtccaagtggtatactgattatgcagtatctgtgaaaagtcgaataaccatcaacccagacacatccaagaaccagttctccctgcagctgaactctgtgactcccgaggacacggctgtgtattactgtgcaagagaatcagagctacgggctgatgcttttgatatctggggccaagggaccacggtcaccgtctcctcaggaggtggcgggtctggtagtggcgctagcggtggtggcggatcctcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaccacagcttccttgaccatcactggggctcaggcggaagatgaggctgactattactgtaactcccgggacagcagtggtaaccatctggtattcggcggagggaccaaggtcaccgtcctaggt

(19) United States
(12) Patent Application Publication
(10) Pub. No.: US 2018/0311374 A1
(43) Pub. Date: Nov. 1, 2018

(54) TARGETED CANCER THERAPY

(71) Applicants: The United States of America, as represented by the Secretary, Department of Health and Human Serv, Bethesda, MD (US); Aleta Biotherapeutics, Inc., Natick, MA (US)

(72) Inventors: Roy Lobb, Westwood, MA (US); Paul David Rennert, Holliston, MA (US); John Todd Schiller, Kensington, MD (US)

(73) Assignees: The United States of America, as represented by the Secretary, Department of Health and Human Serv, Bethesda, MD (US); Aleta Biotherapeutics, Inc., Natick, MA (US)

(21) Appl. No.: 15/772,152

(22) PCT Filed: Oct. 28, 2016
(86) PCT No.: PCT/US16/59388

§ 371 Date: Apr. 30, 2018

Related U.S. Application Data
(60) Provisional application No. 62/249,033, filed on Oct. 30, 2015.

(51) Int. Cl. A61K 47/68; A61K 39/00; A61K 35/76; C07K 16/08; A61K 9/00; A61P 35/00

(52) U.S. Cl. CPC A61K 47/6851; A61K 39/0011; A61K 35/76; A61K 47/6803; A61K 35/17; A61K 9/0019; A61P 35/00; A61K 2039/5158; C07K 16/084

(57) ABSTRACT

Some embodiments of the present disclosure are directed to methods that include delivering to a subject a papillomavirus particle or soluble papillomavirus protein that targets a tumor, and delivering to the subject an immune cell expressing a receptor that binds to a surface antigen of the papillomavirus particle or soluble papillomavirus protein, respectively.

### RELATED APPLICATIONS

This application claims the benefit under 35 U.S.C. § 119(e) of U.S. provisional application No. 62/249,033, filed Oct. 30, 2015, which is incorporated by reference herein in its entirety.

### BACKGROUND

Adoptive cell transfer is a targeted immune cell therapy that often involves engineering a patient's immune cells to recognize and attack his or her tumor(s). Immune cells collected from a patient's blood can be genetically engineered to express receptors on the immune cell surface, which permits recognition by the immune cells of specific ligand proteins (antigens) expressed on a tumor cell surface. In vitro-expanded populations of these genetically-engineered immune cells are infused back into the patient, the immune cells multiply in the patient's body and, with guidance from the engineered receptors, recognize and kill cancer cells that harbor the surface antigen.

### SUMMARY

One approach to immunotherapy involves engineering a patient's own immune cells to recognize and attack his or her tumors. Often, however, the engineered immune cells attack normal cells as well as tumor cells, thus lowering the efficacy of the immunotherapy and increasing unwanted side-effects. This is in part because the tumor cells and the normal cells can express similar surface antigens at different levels. The present disclosure provides compositions and methods for selectively targeting immune cells to tumor cells for the treatment of cancer. This selectively results from engineering (e.g., genetically engineering) tumor cells and immune cells of a subject in a complementary fashion resulting in a highly specific immunotherapeutic targeting system. In some embodiments, the papillomavirus particles comprise virion surface antigens (e.g., papillomavirus-specific antigens) not expressed by normal (non-tumor) cells, while in other embodiments, papillomavirus particles are engineered to comprise non-virion surface antigens not expressed by normal cells or preferentially expressed in tumor cells. These antigens are then selectively bound by immune cells engineered to express cognate receptors (receptors that bind specifically to those antigens).

Some embodiments of the present disclosure provide methods that include delivering to a subject a papillomavirus particle or soluble papillomavirus protein that targets a tumor, and delivering to the subject an immune cell expressing a receptor that binds to a surface antigen of the papillomavirus particle or soluble papillomavirus protein, respectively.

In some embodiments, a papillomavirus particle is a papillomavirus, a papilloma virus-like particle or a papilloma pseudovirus.

In some embodiments, a papillomavirus particle is a human papillomavirus particle, such as a modified human papillomavirus particle. In some embodiments, a papillomavirus particle is a non-human papillomavirus particle. Non-limiting examples of non-human papillomavirus particles include bovine, murine, cottontail-rabbit, macaque or rhesus papillomavirus particles.

In some embodiments, soluble papillomavirus proteins form a capsomer.

In some embodiments, an immune cell is a leukocyte. Non-limiting examples of leukocytes include neutrophils, eosinophils, basophils, lymphocytes and monocytes.

In some embodiments, a leukocyte is a lymphocyte, such as a T cell, a B cell, an NK cell, or an NKT cell. In some embodiments, an immune cell is a dendritic cell.

In some embodiments, a receptor is a recombinant antigen receptor, such as a chimeric antigen receptor.

In some embodiments, a surface antigen of the papillomavirus particle is an L1 protein or an L2 protein.

In some embodiments, a surface antigen is linked (covalently or non-covalently) to a surface of the papillomavirus particle. In some embodiments, a surface antigen is a peptide incorporated into a region of a recombinant capsid protein that is surface-exposed in the papillomavirus particle.

In some embodiments, a surface antigen of the papillomavirus is a self-antigen or a non-self antigen. A non-self antigen may be, for example, a bacterial, yeast, protozoan, viral, plant or fish antigen. In some embodiments, a non-self antigen is a synthetic (artificial) antigen.

In some embodiments, a surface antigen of the papillomavirus is a tumor antigen, such as a tumor-specific antigen (TSA) or a tumor-associated antigen (TAA). In some embodiments, a tumor antigen is or comprises an epitope of CD19, CD20, CD21, CD22, CD45, BCMA, MART-1, MAGE-A3, glycoprotein 100 (gp100), NY-ESO-1, HER2 (ErbB2), IGF2B3, EGFRvIII, Kallikrein 4, KIF20A, Lengsin, Meloe, MUC-1, MUC5AC, MUC-16, B7-H3, B7-H6, CD70, CEA, CSPG4, EphA2, EpCAM, EGFR family, FAP, FRα, glupican-3, GD2, GD3, HLA-A1+MAGE1, IL-11Rα, IL-23Rα2, Lewis-Y, mesothelin, NKG2D ligands, PSMA, ROR1, survivin, TAG72 or VEGFR2.

In some embodiments, a surface antigen of the papillomavirus is or comprises a synthetic epitope. Non-limiting examples of synthetic epitopes include a His tag, a FLAG tag, or an SV5 tag. In some embodiments, a surface antigen of the papillomavirus is or comprises a hapten. Non-limiting examples of haptens include FITC, Alexa-488, LICOR and many others.

In some embodiments, the papillomavirus particle, the immune cell or the papillomavirus particle and the immune cell are delivered via a parenteral, enteric or topical route. In some embodiments, the parenteral route is intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumor, intratympanic, intrauterine, intravascular, intravenous (bolus or drip), intraventricular, intravesical or subcutaneous.

In some embodiments, a tumor is an ocular tumor, a melanoma, a head and neck tumor, a lung tumor, a bladder tumor, a breast tumor, a colorectal tumor, a gastric tumor, an ovarian tumor, a pancreatic tumor, a prostate tumor, a liver tumor, or a renal tumor.

Some embodiments of the present disclosure provide methods that include delivering to a subject a virus that targets a tumor, and delivering to the subject an immune cell comprising a receptor that binds to a surface antigen of the virus.

Other embodiments of the present disclosure provide compositions comprising an immune cell modified to express a tumor-tropic viral protein (e.g., HPV L1) exposed on the immune cell surface.

In some embodiments, the immune cell is leukocyte. For example, the leukocyte may be a neutrophil, eosinophil, basophil, lymphocyte or a monocyte. In some embodiments, the leukocyte is a lymphocyte. The lymphocyte may be, for example, a T cell, a B cell, an NK cell, or an NKT cell. In some embodiments, the immune cell is a dendritic cell.

In some embodiments, the tumor-tropic viral protein is a papilloma protein. In some embodiments, the tumor-tropic viral protein is an HPV L1 protein.

In some embodiments, provided herein are methods comprising delivering to a tumor an immune cell modified to comprise a tumor-tropic viral protein exposed on the immune cell surface. The immune cell may be a leukocyte, for example. In some embodiments, the leukocyte is a neutrophil, eosinophil, basophil, lymphocyte or a monocyte. The leukocyte may be, for example, a lymphocyte. In some embodiments, the lymphocyte is a T cell, a B cell, an NK cell, or an NKT cell. In some embodiments, an immune cell is a dendritic cell.

Some embodiments of the present disclosure provide methods that include delivering to a subject a recombinant protein that targets a tumor, and delivering to the subject an immune cell expressing a receptor that binds to the recombinant protein. In some embodiments, the recombinant protein is a malarial protein. In some embodiments, the malarial protein is a glycosaminoglycan binding protein. In some embodiments, the malarial protein is VAR2CSA (Salanti A et al. Cancer Cell 2015; 28 (4):500-14, incorporated herein by reference).

Also provided herein are methods comprising delivering to a subject an immune cell modified to express a chimeric antigen receptor that comprises an scFv antibody fragment (e.g., a human or humanized antibody fragment) that binds specifically to a papillomavirus surface antigen. In some embodiments, the scFv antibody fragment binds specifically to papillomavirus L1 protein. In some embodiments, the scFv antibody fragment binds specifically to papillomavirus L2 protein. In some embodiments, the immune cell is a T cell.

Further provided herein are methods comprising delivering to a subject an immune cell modified to express a chimeric antigen receptor that comprises a tumor-tropic viral protein. In some embodiments, the tumor-tropic viral protein comprises papillomavirus L1 protein or a L1 peptide capsomer.

Also provided herein are methods comprising delivering to a subject an immune cell modified to express chimeric antigen receptor that comprises an scFv antibody fragment that binds specifically to heparin sulfate proteoglycan (HSPG). In some embodiments, the immune cell is a T cell.

The present disclosure also provided antibodies and antibody fragments (e.g., scFv fragments).

In some embodiments, the present disclosure provides an isolated antibody or antibody fragment comprising: a heavy chain variable domain comprising a CDR1 amino acid sequence that is identical to the amino acid sequence of SEQ ID NO: 4, a CDR2 amino acid sequence that is identical to the amino acid sequence of SEQ ID NO: 5, and a CDR3 amino acid sequence that is identical to the amino acid sequence of SEQ ID NO: 6; and a light chain variable domain comprising a CDR1 amino acid sequence that is identical to the amino acid sequence of SEQ ID NO: 8, a CDR2 amino acid sequence that is identical to the amino acid sequence of SEQ ID NO: 9, and a CDR3 amino acid sequence that is identical to the amino acid sequence of SEQ ID NO: 10.

In some embodiments, at least one framework region amino acid sequence of the heavy chain variable domain is at least 50%, at least 60%, at least 70%, at least 80%, at least 90%, at least 95%, at least 98% or at least 99% identical to at least one corresponding framework region amino acid sequence of a heavy chain variable domain amino acid sequence that is identical to the amino acid sequence of SEQ ID NO: 3. In some embodiments, the isolated antibody or antibody fragment comprises a heavy chain variable domain amino acid sequence that is at least 50%, at least 60%, at least 70%, at least 80%, at least 90%, at least 95%, at least 98% or at least 99% identical to the amino acid sequence of SEQ ID NO: 3. In some embodiments, the isolated antibody or antibody fragment comprises a heavy chain variable domain amino acid sequence that is identical to the amino acid sequence of SEQ ID NO: 3.

In some embodiments, at least one framework region amino acid sequence of the light chain variable domain is at least 50%, at least 60%, at least 70%, at least 80%, at least 90%, at least 95%, at least 98% or at least 99% identical to at least one corresponding framework region amino acid sequence of a light chain variable domain amino acid sequence that is identical to the amino acid sequence of SEQ ID NO: 7. In some embodiments, the isolated antibody or antibody fragment comprises a light chain variable domain amino acid sequence that is at least 50%, at least 60%, at least 70%, at least 80%, at least 90%, at least 95%, at least 98% or at least 99% identical to the amino acid sequence of SEQ ID NO: 7. In some embodiments, the isolated monoclonal antibody comprises a light chain variable domain amino acid sequence that is identical to the amino acid sequence of SEQ ID NO: 7.

In some embodiments, the isolated antibody or antibody fragment comprises a heavy chain variable domain amino acid sequence that is identical to the amino acid sequence of SEQ ID NO: 3 and a light chain variable domain amino acid sequence that is identical to the amino acid sequence of SEQ ID NO: 7.

In some embodiments, the present disclosure provides an isolated antibody or antibody fragment comprising an amino acid sequence identical to SEQ ID NO: 2.

In some embodiments, the present disclosure provides an isolated antibody or antibody fragment encoded by a nucleic acid comprising a nucleic acid identical to SEQ ID NO: 11.

In some embodiments, the present disclosure provides an isolated antibody or antibody fragment comprising: a heavy chain variable domain comprising a CDR1 amino acid sequence that is identical to the amino acid sequence of SEQ ID NO: 14, a CDR2 amino acid sequence that is identical to the amino acid sequence of SEQ ID NO: 15, and a CDR3 amino acid sequence that is identical to the amino acid sequence of SEQ ID NO: 16; and a light chain variable domain comprising a CDR1 amino acid sequence that is identical to the amino acid sequence of SEQ ID NO: 18, a CDR2 amino acid sequence that is identical to the amino acid sequence of SEQ ID NO: 19, and a CDR3 amino acid sequence that is identical to the amino acid sequence of SEQ ID NO: 20.

In some embodiments, at least one framework region amino acid sequence of the heavy chain variable domain is at least 50%, at least 60%, at least 70%, at least 80%, at least 90%, at least 95%, at least 98% or at least 99% identical to at least one corresponding framework region amino acid sequence of a heavy chain variable domain amino acid sequence that is identical to the amino acid sequence of SEQ ID NO: 13. In some embodiments, the isolated antibody or antibody fragment comprises a heavy chain variable domain amino acid sequence that is at least 50%, at least 60%, at least 70%, at least 80%, at least 90%, at least 95%, at least 98% or at least 99% identical to the amino acid sequence of SEQ ID NO: 13. In some embodiments, the isolated antibody or antibody fragment comprises a heavy chain variable domain amino acid sequence that is identical to the amino acid sequence of SEQ ID NO: 13.

In some embodiments, at least one framework region amino acid sequence of the light chain variable domain is at least 50%, at least 60%, at least 70%, at least 80%, at least 90%, at least 95%, at least 98% or at least 99% identical to at least one corresponding framework region amino acid sequence of a light chain variable domain amino acid sequence that is identical to the amino acid sequence of SEQ ID NO: 17. In some embodiments, the isolated antibody or antibody fragment comprises a light chain variable domain amino acid sequence that is at least 50%, at least 60%, at least 70%, at least 80%, at least 90%, at least 95%, at least 98% or at least 99% identical to the amino acid sequence of SEQ ID NO: 17. In some embodiments, the isolated monoclonal antibody comprises a light chain variable domain amino acid sequence that is identical to the amino acid sequence of SEQ ID NO: 17.

In some embodiments, the isolated antibody or antibody fragment comprises a heavy chain variable domain amino acid sequence that is identical to the amino acid sequence of SEQ ID NO: 13 and a light chain variable domain amino acid sequence that is identical to the amino acid sequence of SEQ ID NO: 17.

In some embodiments, the present disclosure provides an isolated antibody or antibody fragment comprising an amino acid sequence identical to SEQ ID NO: 12.

In some embodiments, the present disclosure provides an isolated antibody or antibody fragment encoded by a nucleic acid comprising a nucleic acid identical to SEQ ID NO: 21.

Calculations of “sequence identity” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 depicts an example of a targeted cancer therapy of the present disclosure. In this example, a tumor-tropic papillomavirus (PV) virus-like particle (VLP) or PV capsomer and an immune cell (e.g., a T cell) engineered to express a chimeric antigen receptor (CAR) that comprises a single chain antibody fragment (scFv) that binds specifically to a surface antigen of the PV VLP or PV capsomer are administered to a subject having a tumor/tumor cells. The PV VLP or PV capsomer homes to the tumor cell(s), and the immune cell targets the surface antigen of the PV VLP or PV capsomer.

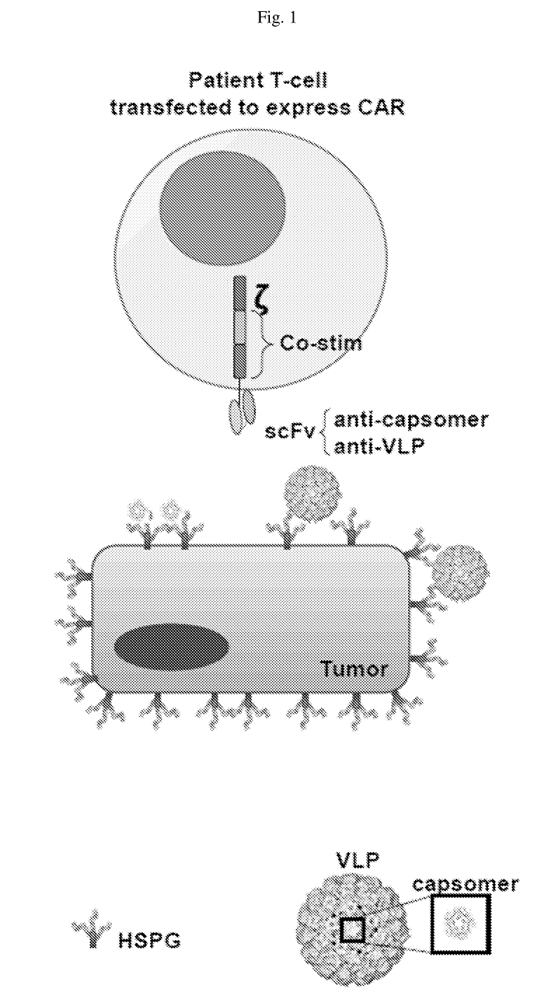

FIG. 2 depicts another example of a targeted cancer therapy of the present disclosure. In this example, an immune cell (e.g., a T cell) is engineered to express a chimeric antigen receptor (CAR) that comprises a tumor-tropic L1 peptide capsomer. The L1 peptide capsomer homes to/binds to HSPGs located on the surface of a tumor/tumor cells. Thus, the engineered immune cell, expressing the tumor-tropic L1 peptide capsomer, is capable of homing to the tumor cell(s).

FIG. 3 depicts yet another example of a targeted cancer therapy of the present disclosure. In this example, an immune cell (e.g., a T cell) is engineered to express a chimeric antigen receptor (CAR) that comprises a scFv that binds specifically to HSPG. The scFv guides the immune cell to HSPGs located on the surface of a tumor/tumor cells. Thus, the engineered immune cell, expressing the scFv, is capable of homing to the tumor cell(s).

FIGS. 4A and 4B show results from an ELISA using HPV scFv #1 and HPV (FIG. 4A) or BVP (FIG. 4B) particles.

FIG. 5 shows results from an ELISA using HPV scFv #2 and HPV particles.

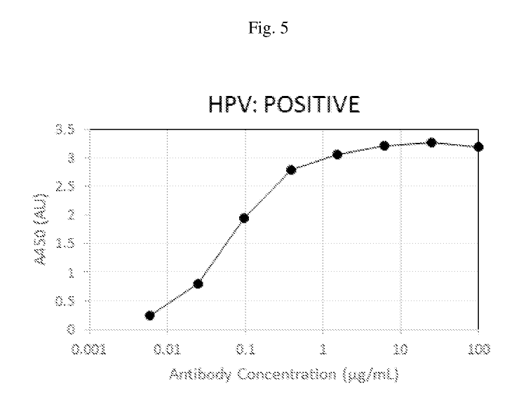

FIG. 6 shows data demonstrating that the scFv antibody fragments described in FIGS. 4, 5A and 5B and 3 bind to human papillomavirus (HPV) particles bound to the surface of tumor cells in vitro.

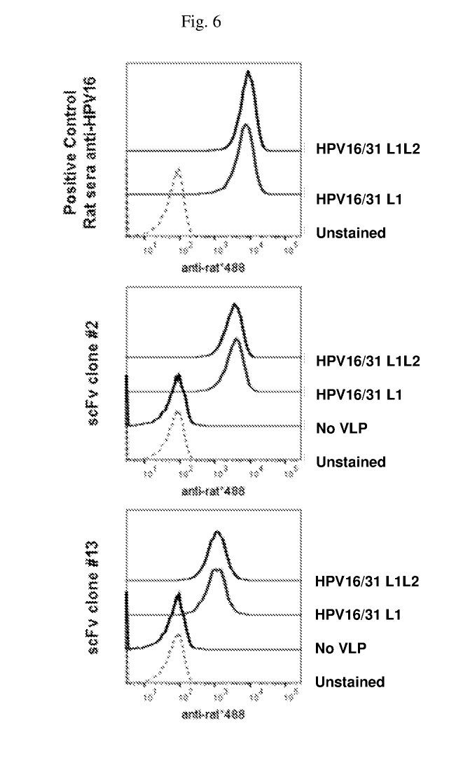

### DETAILED DESCRIPTION

Tumor cells typically express tumor antigens that trigger an immune response in a host subject. These tumor antigens serve as markers for identifying tumor cells and also serve as candidates for targeted cancer therapies. In many instances, however, the antigens expressed by a tumor are also expressed by some normal cells. These antigens are referred to as tumor-associated antigens. Thus, therapies designed to use tumor-associated antigens as signals to guide therapeutics to tumors risk also targeting normal cells, which can result in unwanted side-effects and lower therapeutic efficacy.

Provided herein are therapies used to selectively target tumor cells without also targeting a substantial number of normal cells, thereby reducing or eliminating unwanted side-effects and increasing efficacy of treatment. In some embodiments, tumor-targeting papillomavirus particles that comprise a particular surface antigen are delivered to a subject, and immune cells of the subject genetically engineered to express the cognate receptor that binds to the antigen are also delivered to the subject. The immune cells, guided by receptor-antigen (ligand) binding, selectively target papillomavirus particles that have homed to tumor cells and that comprise the surface antigen (e.g., specific to the papillomavirus particle). The immune cells then kill the tumor cells.

### Papillomavirus Particles

Methods of the present disclosure include delivering to a subject a papillomavirus particle that targets (homes to) tumors and contains one or more surface antigen(s) of interest. The term “papillomavirus particle” encompasses papilloma virus-like particles, papilloma pseudoviruses and soluble papillomavirus protein subunits. In some embodiments, a “papillomavirus particle” is a modified, non-replicating papillomavirus.

“Papillomavirus virus-like particles” (“papillomavirus VLPs”) are organized capsid-like structures (e.g., roughly spherical or cylindrical in shape) that comprise self-assembling ordered arrays of capsomers (aggregates of capsid proteins that self-assemble to form a viral capsid) and do not include a viral genome. A VLP, thus, resembles a virus but is non-infectious because it lacks infectious viral genetic material (DNA or RNA). Papilloma VLPs, generally, are assembled from capsomers containing L1 capsid proteins, or a combination of L1 and L2 capsid proteins, and in some embodiments, surface antigens of interest are conjugated to a capsid protein that forms the papilloma VLP.

“Papilloma pseudoviruses” are synthetic viruses used to transduce (or transfer) into eukaryotic cells genetic material, including DNA and RNA, having specific and desired traits. Pseudoviruses are closely related to viruses in structure and behavior but lack many characteristics exhibited by true viruses, including the capability to replicate. Similar to papillomavirus VLPs, papillomavirus pseudoviruses, generally, are assembled from capsomers containing L1 capsid proteins, or a combination of L1 and L2 capsid proteins, and in some embodiments, surface antigens of interest are conjugated to a capsid protein that forms the papillomavirus pseudovirus.

The term “soluble papillomavirus proteins” are typically soluble capsid proteins and encompass proteins that form pentamers and capsomers comprised of multiple (e.g., five) capsid proteins, including L1 capsid proteins, L2 capsid proteins, or a combination of L1 and L2 capsid proteins.

Papillomavirus particles of the present disclosure may be human, modified human or non-human papillomavirus particles.

Papillomavirus particles are considered “human” if they contain L1 and/or L2 capsid proteins obtained from a human papillomavirus (a papillomavirus that naturally infects humans). Modified human papillomavirus particles include human papillomavirus particles containing capsid proteins that are modified in a way that results in the particle having altered immunogenicity or antigenicity relative to a human papillomavirus particle that comprises or consists of wild-type papillomavirus proteins (wild-type L1 and/or L2 capsid proteins). For example, a modified human papillomavirus particle of the present disclosure may contain a recombinant L1 capsid protein obtained from HPV 16 and HPV 31, referred to as a “modified HPV16/31 L1 protein,” which is described in International Pub. No. WO/2010/120266, the entirety of which is incorporated herein by reference.

Non-limiting examples of non-human papillomavirus particles include bovine, murine, cottontail rabbit, macaque and rhesus monkey papillomavirus particles (e.g., Campo. Vet. J. 1997; 154 (3):175-188; Schulz et al. PLoS One 2012; 7 (10):e47164; Meyers et al. J. of Virology. 1992; 66 (3):1655-1664; Ostrow et al. PNAS. 1990; 87 (20):8170-8174; Gardner and Luciw. ILAR Journal. 2008; 49 (2):220-255). In some embodiments, the non-human papillomavirus particle is a bovine papillomavirus particle (e.g., VLP or pseudovirus).

### Surface Antigens

Some embodiments of the present disclosure are directed to antigens present on or exposed to the surface of a papillomavirus particle. An “antigen” is a molecule that serves as a ligand for receptors of immune cells, including leukocytes, such as T cells. As discussed above, papillomavirus particles typically contain L1 capsid proteins or a combination of L1 and L2 capsid proteins, which form the outermost surface of the particle. Thus, in some embodiments, immune cells of the present disclosure are engineered to express receptors that bind to an L1 capsid protein or an L2 capsid protein (e.g., bind to an epitope of the L1 or L2 capsid protein).

In some embodiments, a papillomavirus particle is engineered to contain an (at least one) antigen that is not a capsid protein, but rather is linked (covalently or non-covalently) to a capsid protein or other particle surface moiety. This antigen (or antigens) may be a self-antigen or a non-self antigen.

A “self-antigen” refers to an antigen that originates from within a body. Self-antigens may be expressed by tumor cells as well as some normal cells. In some embodiments, tumor cells express self-antigens at an expression level higher than the expression level at which a normal tumor cell expresses the same self-antigen. That is, the self-antigen expressed by a tumor cell is overexpressed. It should be understood that while “self-antigens” originate from within the body, a recombinant form of that antigen is still referred to as “self-antigen” if it is linked to a papillomavirus particle.

In some embodiments, a self-antigen is a tumor antigen. A “tumor antigen” is an antigen expressed by tumor cells. Examples of tumor antigens of the present disclosure include, without limitation, CD19, CD20, CD21, CD22, CD45, BCMA, MART-1, MAGE-A3, glycoprotein 100 (gp100), NY-ESO-1, HER2 (ErbB2), IGF2B3, EGFRvIII, Kallikrein 4, KIF20A, Lengsin, Meloe, MUC-1, MUC5AC, MUC-16, B7-H3, B7-H6, CD70, CEA, CSPG4, EphA2, EpCAM, EGFR family, FAP, FRα, glupican-3, GD2, GD3, HLA-A1+MAGE1, IL-11Rα, IL-23Rα2, Lewis-Y, mesothelin, NKG2D ligands, PSMA, ROR1, survivin, TAG72 or VEGFR2. Other examples of tumor antigens are described (der Bruggen P et al. Peptide database: T cell-defined tumor antigens. Cancer Immun 2013. URL: cancerimmunity.org/peptide, incorporated herein by reference).

Tumor antigens include tumor-specific antigens (TSA) and tumor-associated antigens (TAA). “Tumor-specific antigens” are expressed only by tumor cells (not expressed on any other cell). “Tumor-associated antigens” are expressed by tumor cells and by some normal (non-tumor) cells.

Examples of tumor antigens include, without limitation, alpha-actinin-4, ARTC1, BCR-ABL, B-RAF, CASP-5, CASP-8, beta-catenin, Cdc27, CDK4, CDK12, CDKN2A, CLPP, COA-1, CSNK1A1, dek-can, EFTUD2, Elongation factor 2, ETV6-AML1, FLT3-ITD, FN1, GAS7, GPNMB, HAUS3, LDLR-fucosyltransferaseAS, HLA-A2, HLA-A11, hsp70-2, MART2, MATN, ME1, MUM-1, MUM-2, MUM-3, neo-PAP, Myosin class I, NFYC, OGT, OS-9, p53, pmI-RARalpha, PPP1R3B, PRDX5, PTPRK, K-ras, N-ras, RBAF600, SIRT2, SNRPD1, SYT-SSX1, SYT-SSX2, TGF-betaRII, Triosephosphate isomerase, BAGE family antigens, CAGE family antigens, Cyclin-A1, GAGE family antigens, HERV-K-MEL, KK-LC-1, KM-HN-1, LAGE-1, MAGE family antigens, NA88-A, NY-ESO-1/LAGE-2, PRAME, SAGE family antigens, Sp17, SSX family antigens, TAG-1, TAG-2, TRAG-3, TRP2-INT2, XAGE family antigens, CEA, Gp100/pmel17, mammaglobin-A, Melan-A/MART-1, mesothelin, NY-BR-1, OA1, PAP, PSA, RAB38/NY-MEL-1, TRP-1/gp75, TRP-2, tyrosinase, 9D7, adipophilin, AIM-2, ALDH1A1, BCLX (L), BING-4, CALCA, CD45, CD274, CPSF, cyclin-B1, cyclin D1, DKK1, ENAH (hMena), EpCAM, EphA3, EZH2, FGF5, Ganglioside GD3, glypican-3, G250/MN/CAIX, HER-2/neu, HLA-DOB, Hepsin, ISO1, IGF2B3, IL13Ralpha2, Intestinal carboxyl esterase, alpha-foetoprotein, Kallikrein 4, KIF20A, Lengsin, M-CSF, MCSP, mdm-2, Meloe, Midkine, MMP-2, MMP-7, MUC1, MUC5AC, p53, PAX5, PBF, PRAME, PSMA, RAGE-1, RGS5, RhoC, RNF43, RU2AS, SAP-1, secernin 1, SOX10, STEAP1, survivin, Telomerase, TPBG, VEGF and WT1. Other tumor antigens are encompassed by the present disclosure.

A “non-self antigen” is an antigen that originates from the external environment (outside the body). A non-self antigen is not naturally expressed in cells (normal cells or tumor cells) of a subject. With respect to a human subject, a non-self antigen may be, for example, a human antigen obtained from a different host/subject or a non-human antigen, such as a bacterial antigen, a yeast antigen, a protozoan antigen, a viral antigen. A non-self antigen may be a naturally-occurring antigen (naturally-occurring in another organisms) or a synthetic (non-naturally-occurring, e.g., artificial) antigen. Examples of non-self antigens include, without limitation, green fluorescent protein, KLH and avian ovalbumin.

### Delivery Routes

In some embodiments, papillomavirus particles, immune cells, or both, are delivered to a subject via a parenteral route, an enteral route or a topical route.

Examples of parental routes include, without limitation, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratympanic, intrauterine, intravascular, intravenous (bolus or drip), intraventricular, intravesical and subcutaneous.

Enteral routes of administration include administration to the gastrointestinal tract via the mouth (oral), stomach (gastric) and rectum (rectal). Gastric administration typically involves the use of a tube through the nasal passage (NG tube) or a tube in the belly leading directly to the stomach (PEG tube). Rectal administration typically involves rectal suppositories.

Topical routes of administration include administration to a body surface, such as skin or mucous membranes. Papillomavirus particles and/or immune cells of the present disclosure may be administered topically via a cream, foam, gel, lotion or ointment, for example.

Other routes of delivery are encompassed by the present disclosure. For example, papillomavirus particles and/or immune cells may be delivered via ultrasound-targeted microbubble destruction (UTMD) (Qiu L. et al. 2012 Gene Therapy 19: 703-710, incorporated herein by reference).

In some embodiments, papillomavirus particles are delivered to a subject prior to or after delivering an immune cell. Thus, a papillomavirus particle and an immune cell may be delivered sequentially. In other embodiments, however, a papillomavirus particle and an immune cell are delivered simultaneously.

### Immune Cells

Some embodiments of the present disclosure are directed to immune cells, such as leukocytes (nucleated white blood cells), comprising (e.g., expressing) a receptor that binds to an antigen. A leukocyte of the present disclosure may be, for example, a neutrophil, eosinophil, basophil, lymphocyte or a monocyte. In some embodiments, a leukocyte is a lymphocyte. Examples of lymphocytes include T cells, B cells, Natural Killer (NK) cells or NKT cells. In some embodiments, a T cell is a CD4⁺ Th (T helper) cell, a CD8⁺ cytotoxic T cell, a γδ T cell or a regulatory (suppressor) T cell. In some embodiments, an immune cell is a dendritic cell.

Immune cells of the present disclosure, in some embodiments, are genetically engineered to express an antigen-binding receptor. A cell is considered “engineered” if it contains an engineered (exogenous) nucleic acid. Engineered nucleic acids of the present disclosure may be introduced into a cell by any known (e.g., conventional) method. For example, an engineered nucleic acid may be introduced into a cell by electroporation (see, e.g., Heiser W. C. Transcription Factor Protocols: Methods in Molecular Biology™ 2000; 130: 117-134), chemical (e.g., calcium phosphate or lipid), transfection (see, e.g., Lewis W. H., et al., Somatic Cell Genet. 1980 May; 6 (3): 333-47; Chen C., et al., Mol Cell Biol. 1987 August; 7 (8): 2745-2752), fusion with bacterial protoplasts containing recombinant plasmids (see, e.g., Schaffner W. Proc Natl Acad Sci USA. 1980 April; 77 (4): 2163-7), microinjection of purified DNA directly into the nucleus of the cell (see, e.g., Capecchi M. R. Cell. 1980 November; 22 (2 Pt 2): 479-88), or retrovirus transduction.

Some aspects of the present disclosure provide an “adoptive cell” approach, which involves isolating immune cells (e.g., T cells) from a subject, genetically engineering the cells (e.g., to express an antigen-binding receptor, such as a chimeric antigen receptor), expanding the cells ex vivo, and then re-introducing the cells into the subject. This method results in a greater number of engineered immune cells in the subject relative to what could be achieved by conventional gene delivery and vaccination methods. In some embodiments, immune cells are isolated from a subject, expanded ex vivo without genetic modification, and then re-introduced into the subject.

### Cell Receptors

Immune cells of the present disclosure comprise receptors that bind to antigens, such as an antigen present on or exposed to the surface of a papillomavirus particle, as provided herein. In some embodiments, a leukocyte is modified (e.g., genetically modified) to express a receptor that binds to an antigen. The receptor may be, in some embodiments, a naturally-occurring antigen receptor (normally expressed on the immune cell) or recombinant antigen receptor (not normally expressed on the immune cell), including, for example, a chimeric antigen receptor (CAR). Naturally-occurring and recombinant antigen receptors encompassed by the present disclosure include T cell receptors, B cell receptors, NK cell receptors, NKT cell receptors and dendritic cell receptors.

Synthetic epitopes can be derived from surface antigens (Matiu et al., J. Immunology. 1983; 130 (4):1947-1952). In some embodiments, the surface antigen comprises a synthetic epitope. In some embodiments, the synthetic epitope comprises a HIS tag, FLAG tag, or SV5 tag. Examples of other tags that may be used herein include viral peptides (e.g., CMV peptides, SV5 peptides), chitin binding protein, maltose binding protein, glutathione-S-transferase, thioredoxin, poly(NANP), Myc-tag, HA-tag, AviTag, calmodulin-tag, polyglutamate tag, E-tag, S-tag, SBP-tag, Softag 1, Strep-tag, TC tag, V5 tag, VSV tag, Xpress tag, isopeptag, Spytag, BCCP, Halo-tag, Nus-tag, Fc-tag and Ty tag. Other tags, for example haptens, are encompassed by the present disclosure.

A “chimeric antigen receptor” refers to an artificial immune cell receptor that is engineered to recognize and bind to an antigen expressed by tumor cells. Generally, a CAR is designed for a T cell and is a chimera of a signaling domain of the T-cell receptor (TcR) complex and an antigen-recognizing domain (e.g., a single chain fragment (scFv) of an antibody or other antibody fragment) (Enblad et al., Human Gene Therapy. 2015; 26 (8):498-505).

In some embodiments, an antigen binding receptor is a chimeric antigen receptor (CAR). A T cell that expressed a CAR is referred to as a “CAR T cell.” A CAR T cell receptor, in some embodiments, comprises a signaling domain of the T-cell receptor (TcR) complex and an antigen-recognizing domain (e.g., a single chain fragment (scFv) of an antibody) (Enblad et al., Human Gene Therapy. 2015; 26 (8):498-505).

There are four generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TcR CD3-ζ chain. Third-generation costimulatory domains may include, e.g., a combination of CD3z, CD27, CD28, 4-1BB, ICOS, or OX40. CARs, in some embodiments, contain an ectodomain (e.g., CD3ζ), commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood. 2015; 125 (26):4017-4023; Kakarla and Gottschalk, Cancer J. 2014; 20 (2):151-155).

In some embodiments, the chimeric antigen receptor (CAR) is a T-cell redirected for universal cytokine killing (TRUCK), also known as a fourth generation CAR. TRUCKs are CAR-redirected T-cells used as vehicles to produce and release a transgenic cytokine, IL-12, that accumulates in the targeted tissue, e.g., a targeted tumor tissue. The transgenic cytokine is released upon CAR engagement of the target. This may result in therapeutic concentrations at the targeted site and avoid systemic toxicity.

CARs typically differ in their functional properties. The CD3ζ signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs, but preclinical in vivo studies suggest that 4-1BB CARs may produce superior proliferation and/or persistence. Clinical trials suggest that both of these second-generation CARs are capable of inducing substantial T-cell proliferation in vivo, but CARs containing the 4-1BB costimulatory domain appear to persist longer. Third generation CARs combine multiple signaling domains (costimulatory) to augment potency. Fourth generation CARs are additionally modified with a constitutive or inducible expression cassette for a transgenic cytokine, which is released by the CAR T-cell to modulate the T-cell response. See, for example, Enblad et al., Human Gene Therapy. 2015; 26 (8):498-505; Chmielewski and Hinrich, Expert Opinion on Biological Therapy. 2015; 15 (8): 1145-1154.

In some embodiments, a chimeric antigen receptor is a first generation CAR. In some embodiments, a chimeric antigen receptor is a third generation CAR. In some embodiments, a chimeric antigen receptor is a second generation CAR. In some embodiments, a chimeric antigen receptor is a third generation CAR.

In some embodiments, a chimeric antigen receptor (CAR) comprises an extracellular domain comprising an antigen binding domain, a transmembrane domain, and a cytoplasmic domain. In some embodiments, a CAR is fully human. In some embodiments, the antigen binding domain of a CAR is specific for one or more antigens. In some embodiments, a “spacer” domain or “hinge” domain is located between an extracellular domain (comprising the antigen binding domain) and a transmembrane domain of a CAR, or between a cytoplasmic domain and a transmembrane domain of the CAR. A “spacer domain” refers to any oligopeptide or polypeptide that functions to link the transmembrane domain to the extracellular domain and/or the cytoplasmic domain in the polypeptide chain. A “hinge domain” refers to any oligopeptide or polypeptide that functions to provide flexibility to the CAR, or domains thereof, or to prevent steric hindrance of the CAR, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of a CAR.

In some embodiments, a CAR of the disclosure comprises an antigen binding domain, such as a single chain Fv (scFv) specific for a tumor antigen. The choice of binding domain depends upon the type and number of ligands that define the surface of a target cell. For example, the antigen binding domain may be chosen to recognize a ligand that acts as a cell surface marker on target cells associated with a particular disease state, such as cancer or an autoimmune disease. Thus, examples of cell surface markers that may act as ligands for the antigen binding domain in the CAR of the present disclosure include those associated with cancer cells and/or other forms of diseased cells. In some embodiments, a CAR is engineered to target a tumor antigen of interest by way of engineering a desired antigen binding domain that specifically binds to a surface antigen of a papillomavirus particle.

An antigen binding domain (e.g., an scFV) that “specifically binds” to a target or an epitope is a term understood in the art, and methods to determine such specific binding are also known in the art. A molecule is said to exhibit “specific binding” if it reacts or associates more frequently, more rapidly, with greater duration and/or with greater affinity with a particular target antigen than it does with alternative targets. An antigen binding domain (e.g., an scFV) that specifically binds to a first target antigen may or may not specifically bind to a second target antigen. As such, “specific binding” does not necessarily require (although it can include) exclusive binding.

In some embodiments, immune cells expressing a CAR are genetically modified to recognize multiple targets or antigens, which permits the recognition of unique target or antigen expression patterns on tumor cells. Examples of CARs that can bind multiple targets include: “split signal CARs,” which limit complete immune cell activation to tumors expressing multiple antigens; “tandem CARs” (TanCARs), which contain ectodomains having two scFvs; and “universal ectodomain CARs,” which incorporate avidin or a fluorescein isothiocyanate (FITC)-specific scFv to recognize tumor cells that have been incubated with tagged monoclonal antibodies (Mabs).

A CAR is considered “bispecific” if it recognizes two distinct antigens (has two distinct antigen recognition domains). In some embodiments, a bispecific CAR is comprised of two distinct antigen recognition domains present in tandem on a single transgenic receptor (referred to as a TanCAR; see, e.g., Grada Z et al. Molecular Therapy Nucleic Acids 2013; 2:e105, incorporated herein by reference). In some embodiments, a bispecific CAR recognizes an L1 protein and a tumor antigen.

In some embodiments, a CAR is an antigen-specific inhibitory CAR (iCAR), which may be used, for example, to avoid off-tumor toxicity (Fedorov, V D et al. Sci. Transl. Med. published online Dec. 11, 2013, incorporated herein by reference). iCARs contain an antigen-specific inhibitory receptor, for example, to block nonspecific immunosuppression, which may result from extratumor target expression. iCARs may be based, for example, on inhibitory molecules CTLA-4 or PD-1, to block immunosuppression, or on a pan-leukocyte antigen, such as CD52, to block leukocyte destruction. In some embodiments, these iCARs block T cell responses from T cells activated by either their endogenous T cell receptor or an activating CAR. In some embodiments, this inhibiting effect is temporary.

In some embodiments, CARs may be used in adoptive cell transfer, wherein immune cells are removed from a subject and modified so that they express receptors specific to an antigen, e.g., a tumor-specific antigen. The modified immune cells, which may then recognize and kill the cancer cells, are reintroduced into the subject (Pule, et al., Cytotherapy. 2003; 5 (3): 211-226; Maude et al., Blood. 2015; 125 (26): 4017-4023, each of which is incorporated herein by reference).

In some embodiments, the antigen binding domain of a CAR is a HPV scFV. For example, the antigen binding domain of a CAR may be a HPV scFv comprising: a heavy chain variable domain comprising a CDR1 amino acid sequence that is identical to the amino acid sequence of SEQ ID NO: 4, a CDR2 amino acid sequence that is identical to the amino acid sequence of SEQ ID NO: 5, and a CDR3 amino acid sequence that is identical to the amino acid sequence of SEQ ID NO: 6; and a light chain variable domain comprising a CDR1 amino acid sequence that is identical to the amino acid sequence of SEQ ID NO: 8, a CDR2 amino acid sequence that is identical to the amino acid sequence of SEQ ID NO: 9, and a CDR3 amino acid sequence that is identical to the amino acid sequence of SEQ ID NO: 10.

In some embodiments, the antigen binding domain of a CAR is a HPV scFv comprising a heavy chain variable domain amino acid sequence that is identical to the amino acid sequence of SEQ ID NO: 3 and a light chain variable domain amino acid sequence that is identical to the amino acid sequence of SEQ ID NO: 7.

In some embodiments, antigen binding domain of a CAR is a HPV scFv comprising an amino acid sequence identical to SEQ ID NO: 2.

In some embodiments, the antigen binding domain of a CAR is a HPV scFv encoded by a nucleic acid comprising a nucleic acid identical to SEQ ID NO: 11.

In some embodiments, the antigen binding domain of a CAR is a HPV scFv comprising: a heavy chain variable domain comprising a CDR1 amino acid sequence that is identical to the amino acid sequence of SEQ ID NO: 14, a CDR2 amino acid sequence that is identical to the amino acid sequence of SEQ ID NO: 15, and a CDR3 amino acid sequence that is identical to the amino acid sequence of SEQ ID NO: 16; and a light chain variable domain comprising a CDR1 amino acid sequence that is identical to the amino acid sequence of SEQ ID NO: 18, a CDR2 amino acid sequence that is identical to the amino acid sequence of SEQ ID NO: 19, and a CDR3 amino acid sequence that is identical to the amino acid sequence of SEQ ID NO: 20.

In some embodiments, the antigen binding domain of a CAR is a HPV scFv comprising a heavy chain variable domain amino acid sequence that is identical to the amino acid sequence of SEQ ID NO: 13 and a light chain variable domain amino acid sequence that is identical to the amino acid sequence of SEQ ID NO: 17.

In some embodiments, the antigen binding domain of a CAR is a HPV scFv comprising an amino acid sequence identical to SEQ ID NO: 12.

In some embodiments, the antigen binding domain of a CAR is a HPV scFv comprising a nucleic acid identical to SEQ ID NO: 21.

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a beta-sheet conformation and the CDRs form loops which connect, and in some cases form part of, the beta-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In some embodiments, an antibody or antibody fragment (e.g., scFv) is humanized. An antibody, or antibody fragment, in some embodiments, may be fully human. In some embodiments, an antibody, or antibody fragment, is chimeric. Antibodies, and antibody fragments, may be monoclonal or polyclonal.

### Tumor Cells

The present disclosure encompasses the treatment of all types of tumors, including primary tumors and metastatic tumors. Tumors that arise from connective tissue, endothelium, mesothelium, blood cells, lymphoid cells, muscle, epithelial tissue, neural tissue and neural crest-derived cells are encompassed herein. The present disclosure also encompasses carcinomas, sarcomas, myelomas, leukemias, lymphomas, and cancers of mixed type (e.g., adenosquamous, carcinoma, mixed mesodermal tumor, carcinosarcoma and teratocarcinoma).

The following is a list of non-limiting examples of tumors/cancers encompassed by the present disclosure: acute lymphoblastic leukemia (ALL), acute myeloid leukemia (AML), adrenocortical carcinoma, AIDS-related cancers, Kaposi sarcoma, AIDS-related lymphoma, primary CNS lymphoma, anal cancer, appendix cancer, astrocytomas, atypical teratoid/rhabdoid tumor, basal cell carcinoma, bile duct cancer, bladder cancer, bone cancer, Ewing sarcoma family of tumors, osteosarcoma and malignant fibrous histiocytoma, brain stem glioma, brain tumor, astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, Burkitt lymphoma, carcinoid tumor, gastrointestinal, carcinoma of unknown primary, cardiac (heart) tumors, atypical teratoid/rhabdoid tumor, embryonal tumors, germ cell tumor, primary lymphoma, cervical cancer, cholangiocarcinoma, chordoma, chronic lymphocytic leukemia (CLL), chronic myelogenous leukemia (CML), chronic myeloproliferative neoplasms, colon cancer, colorectal cancer, craniopharyngioma, cutaneous T-cell lymphoma, ductal carcinoma in situ (DCIS), embryonal tumors, central nervous system, endometrial cancer, ependymoma, esophageal cancer, esthesioneuroblastoma, ewing sarcoma, extracranial germ cell tumor, extragonadal germ cell tumor, eye cancer, intraocular melanoma, retinoblastoma, fallopian tube cancer, fibrous histiocytoma of bone, malignant, and osteosarcoma, gallbladder cancer, gastric (stomach) cancer, gastrointestinal carcinoid tumor, gastrointestinal stromal tumors (gist), germ cell tumor, central nervous system, extracranial, extragonadal, ovarian, testicular, gestational trophoblastic disease, glioma, brain stem, hairy cell leukemia, head and neck cancer, heart cancer, hepatocellular (liver) cancer, histiocytosis, langerhans cell, Hodgkin lymphoma, hypopharyngeal cancer, intraocular melanoma, islet cell tumors, pancreatic neuroendocrine tumors, kaposi sarcoma, kidney, renal cell, Wilms tumor and other kidney tumors, langerhans cell histiocytosis, laryngeal cancer, acute lymphoblastic leukemia (ALL), acute myeloid leukemia (AML), chronic lymphocytic leukemia (CLL), chronic myeloid leukemia (CML), hairy cell, lip and oral cavity cancer, liver cancer (primary), lung cancer, non-small cell, small cell, lymphoma, Burkitt, cutaneous t-cell, Hodgkin, non-Hodgkin, primary central nervous system (CNS), macroglobulinemia, waldenström, male breast cancer, malignant fibrous histiocytoma of bone and osteosarcoma, melanoma, intraocular (eye), merkel cell carcinoma, mesothelioma, malignant, metastatic squamous neck cancer with occult primary, midline tract carcinoma involving nut gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, myeloma, myeloproliferative neoplasms, chronic, nasal cavity and paranasal sinus cancer, nasopharyngeal cancer, neuroblastoma, non-Hodgkin lymphoma, non-small cell lung cancer, ocular, oral cancer, oral cavity cancer, lip and, oropharyngeal cancer, osteosarcoma and malignant fibrous histiocytoma of bone, ovarian cancer, epithelial, germ cell tumor, low malignant potential tumor, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, renal cell (kidney) cancer, renal pelvis and ureter, transitional cell cancer, retinal cancer, retinoblastoma, rhabdomyosarcoma, salivary gland cancer, sarcoma, ewing, kaposi, osteosarcoma (bone cancer), rhabdomyosarcoma, soft tissue, uterine, Sézary syndrome, skin cancer, melanoma, merkel cell carcinoma, nonmelanoma, small cell lung cancer, small intestine cancer, soft tissue sarcoma, squamous cell carcinoma, squamous neck cancer with occult primary, metastatic, stomach (gastric) cancer, t-cell lymphoma, cutaneous, testicular cancer, throat cancer, thymoma and thymic carcinoma, thyroid cancer, transitional cell cancer of the renal pelvis and ureter, unknown primary, carcinoma of, unusual cancers of, ureter and renal pelvis, transitional cell cancer, urethral cancer, uterine cancer, endometrial, uterine sarcoma, vaginal cancer, vulvar cancer and waldenström macroglobulinemia.

### Nucleic Acids

Some embodiments of the present disclosure provide immune cells that are engineered to express a receptor that binds to a surface antigen of a papillomavirus particle. As indicated above, an immune cell is considered “engineered” if it contains an engineered nucleic acid. An “engineered nucleic acid” is a nucleic acid (e.g., at least two nucleotides covalently linked together, and in some instances, containing phosphodiester bonds, referred to as a phosphodiester “backbone”) that does not occur in nature. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with (also referred to as “binding to,” e.g., transiently or stably) naturally-occurring nucleic acid molecules. Recombinant and synthetic nucleic acids also include those molecules that result from the replication of either of the foregoing.

While an engineered nucleic acid, as a whole, is not naturally-occurring, it may include wild-type nucleotide sequences. In some embodiments, an engineered nucleic acid comprises nucleotide sequences obtained from different organisms (e.g., obtained from different species). For example, in some embodiments, an engineered nucleic acid includes a murine nucleotide sequence, a bacterial nucleotide sequence, a human nucleotide sequence, a viral nucleotide sequence, or a combination of any two or more of the foregoing sequences.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

### Additional Embodiments

Additional embodiments of the present disclosure are encompassed by the following number paragraphs:

1. A method comprising delivering to a subject a papillomavirus particle or soluble papillomavirus protein that targets a tumor, and delivering to the subject an immune cell expressing a receptor that binds to a surface antigen of the papillomavirus particle or soluble papillomavirus protein, respectively.
2. The method of paragraph 1, wherein the papillomavirus particle is a papilloma virus-like particle or a papilloma pseudovirus.
3. The method of paragraph 1 or 2, wherein the papillomavirus particle or soluble papillomavirus protein is a human papillomavirus particle or a soluble human papillomavirus protein.
4. The method of paragraph 1 or 2, wherein the papillomavirus particle or soluble papillomavirus protein is a modified human papillomavirus particle or a soluble modified human papillomavirus protein.
5. The method of paragraph 1 or 2, wherein the papillomavirus particle or soluble papillomavirus protein is a non-human papillomavirus particle or a soluble non-human papillomavirus protein.
6. The method of paragraph 4, wherein the non-human papillomavirus particle is a bovine, murine, cotton-rabbit, macaque or rhesus papillomavirus particle or soluble papillomavirus protein.
7. The method of paragraph 6, wherein the non-human papillomavirus particle or soluble non-human papillomavirus protein is a bovine papillomavirus particle or soluble bovine papillomavirus protein.
8. The method of any one of paragraphs 1-6, wherein the papillomavirus particle is a papilloma virus-like particle.
9. The method of any one of paragraphs 1-6, wherein the papillomavirus particle is a papilloma pseudovirus.
10. The method of any one of paragraphs 1-6, wherein the soluble papillomavirus protein forms a capsomer.
11. The method of paragraph 1, wherein the capsomer comprises of L1 protein.
12. The method of any one of paragraphs 1-11, wherein the immune cell is leukocyte.
13. The method of paragraph 12, wherein the leukocyte is a neutrophil, eosinophil, basophil, lymphocyte or a monocyte.
14. The method of paragraph 13, wherein the leukocyte is a lymphocyte.
15. The method of paragraph 14, wherein the lymphocyte is a T cell, a B cell, an NK cell, or an NKT cell.
16. The method of paragraph 15, wherein the lymphocyte is a T cell.
17. The method of any one of paragraphs 1-11, wherein immune cell is a dendritic cell.
18. The method of any one of paragraphs 1-17, wherein the receptor is a recombinant antigen receptor.
19. The method of any one of paragraphs 1-17, wherein the receptor is a chimeric antigen receptor.
20. The method of any one of paragraphs 1-19, wherein the surface antigen of the papillomavirus particle is an L1 protein or an L1/L2 protein complex.
21. The method of any one of paragraphs 1-20, wherein the surface antigen is covalently linked to a surface of the papillomavirus particle.
22. The method of paragraph 21, wherein the surface antigen is a hapten.
23. The method of any one of paragraphs 1-22, wherein the surface antigen is non-covalently linked to a surface of the papillomavirus particle.
24. The method of any one of paragraphs 1-20, wherein the surface antigen is a peptide incorporated into a region of a recombinant capsid protein that is surface-exposed in the papillomavirus particle.
25. The method of any one of paragraphs 1-24, wherein the surface antigen of the papillomavirus is a self-antigen.
26. The method of any one of paragraphs 1-24, wherein the surface antigen of the papillomavirus is a non-self antigen.
27. The method of paragraph 26, wherein the non-self antigen is a bacterial, yeast, protozoan or viral antigen.
28. The method of any one of paragraphs 1-24, wherein the surface antigen of the papillomavirus is a tumor antigen.
29. The method of paragraph 28, wherein the tumor antigen is a tumor-specific antigen (TSA) or a tumor-associated antigen (TAA).
30. The method of paragraph 29, wherein the tumor antigen is or comprises an epitope of CD19, CD20, CD21, CD22, CD45, BCMA, MART-1, MAGE-A3, glycoprotein 100 (gp100), NY-ESO-1, HER2 (ErbB2), IGF2B3, EGFRvIII, Kallikrein 4, KIF20A, Lengsin, Meloe, MUC-1, MUC5AC, MUC-16, B7-H3, B7-H6, CD70, CEA, CSPG4, EphA2, EpCAM, EGFR family, FAP, FRα, glupican-3, GD2, GD3, HLA-A1+MAGE1, IL-11Rα, IL-23Rα2, Lewis-Y, mesothelin, NKG2D ligands, PSMA, ROR1, survivin, TAG72 or VEGFR2.
31. The method of paragraph 30, wherein the tumor antigen is or comprises an epitope of CD19.
32. The method of paragraph 30, wherein the tumor antigen is selected from full length CD19, a fragment of CD19, at least one C2 Ig-like domain of CD19, or a linear epitope of CD19.
33. The method of any one of paragraphs 1-24, wherein the surface antigen of the papillomavirus is or comprises a synthetic epitope.
34. The method of paragraph 33, wherein the synthetic epitope is a His tag, a FLAG tag, or an SV5 tag.
35. The method of any one of paragraphs 1-34, wherein the papillomavirus particle, the immune cell or the papillomavirus particle and the immune cell are delivered via a parenteral, enteric or topical route.
36. The method of paragraph 35, wherein the parenteral route is intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumor, intratympanic, intrauterine, intravascular, intravenous (bolus or drip), intraventricular, intravesical or subcutaneous.
37. The method of any one of paragraphs 1-36, wherein the tumor is an ocular tumor, a melanoma, a head and neck tumor, a lung tumor, a bladder tumor, a breast tumor, a colorectal tumor, a gastric tumor, an ovarian tumor, a pancreatic tumor, a prostate tumor, a liver tumor, a renal tumor, or mesothelioma].
38. A method comprising delivering to a subject a virus that targets a tumor, and delivering to the subject an immune cell comprising a receptor that binds to a surface antigen of the virus.
39. A method comprising delivering to a subject an immune cell modified to express a chimeric antigen receptor that comprises an scFv antibody fragment that binds specifically to a papillomavirus surface antigen.
40. The method of paragraph 39, wherein the scFv antibody fragment binds specifically to papillomavirus L1 protein.
41. A method comprising delivering to a subject an immune cell modified to express a chimeric antigen receptor that comprises a tumor-tropic viral protein.
42. The method of paragraph 41, wherein the tumor-tropic viral protein comprises papillomavirus L1 protein or a L1 peptide capsomer.
43. A method comprising delivering to a subject an immune cell modified to express chimeric antigen receptor that comprises an scFv antibody fragment that binds specifically to heparin sulfate proteoglycan (HSPG).
44. The method of any one of paragraphs 39-43, wherein the immune cell is a T cell.
45. An isolated antibody or antibody fragment comprising:

a heavy chain variable domain comprising a CDR1 amino acid sequence that is identical to the amino acid sequence of SEQ ID NO: 4, a CDR2 amino acid sequence that is identical to the amino acid sequence of SEQ ID NO: 5, and a CDR3 amino acid sequence that is identical to the amino acid sequence of SEQ ID NO: 6; and

a light chain variable domain comprising a CDR1 amino acid sequence that is identical to the amino acid sequence of SEQ ID NO: 8, a CDR2 amino acid sequence that is identical to the amino acid sequence of SEQ ID NO: 9, and a CDR3 amino acid sequence that is identical to the amino acid sequence of SEQ ID NO: 10.

46. The isolated antibody or antibody fragment of paragraph 45, wherein at least one framework region amino acid sequence of the heavy chain variable domain is at least 90% identical to at least one corresponding framework region amino acid sequence of a heavy chain variable domain amino acid sequence that is identical to the amino acid sequence of SEQ ID NO: 3.
47. The isolated antibody or antibody fragment of paragraph 45, wherein the isolated antibody or antibody fragment comprises a heavy chain variable domain amino acid sequence that is at least 90% identical to the amino acid sequence of SEQ ID NO: 3.
48. The isolated antibody or antibody fragment of paragraph 47, wherein the isolated antibody or antibody fragment comprises a heavy chain variable domain amino acid sequence that is identical to the amino acid sequence of SEQ ID NO: 3.
49. The isolated antibody or antibody fragment of paragraph 45, wherein at least one framework region amino acid sequence of the light chain variable domain is at least 90% identical to at least one corresponding framework region amino acid sequence of a light chain variable domain amino acid sequence that is identical to the amino acid sequence of SEQ ID NO: 7.
50. The isolated antibody or antibody fragment of paragraph 45, wherein the isolated antibody or antibody fragment comprises a light chain variable domain amino acid sequence that is at least 90% identical to the amino acid sequence of SEQ ID NO: 7.
51. The isolated antibody or antibody fragment of paragraph 50, wherein the isolated monoclonal antibody comprises a light chain variable domain amino acid sequence that is identical to the amino acid sequence of SEQ ID NO: 7.
52. The isolated antibody or antibody fragment of paragraph 45, wherein the isolated antibody or antibody fragment comprises a heavy chain variable domain amino acid sequence that is identical to the amino acid sequence of SEQ ID NO: 3 and a light chain variable domain amino acid sequence that is identical to the amino acid sequence of SEQ ID NO: 7.
53. An isolated antibody or antibody fragment comprising an amino acid sequence identical to SEQ ID NO: 2.
54. An isolated antibody or antibody fragment encoded by a nucleic acid comprising a nucleic acid identical to SEQ ID NO: 11.
55. An isolated antibody or antibody fragment comprising:

a heavy chain variable domain comprising a CDR1 amino acid sequence that is identical to the amino acid sequence of SEQ ID NO: 14, a CDR2 amino acid sequence that is identical to the amino acid sequence of SEQ ID NO: 15, and a CDR3 amino acid sequence that is identical to the amino acid sequence of SEQ ID NO: 16; and

a light chain variable domain comprising a CDR1 amino acid sequence that is identical to the amino acid sequence of SEQ ID NO: 18, a CDR2 amino acid sequence that is identical to the amino acid sequence of SEQ ID NO: 19, and a CDR3 amino acid sequence that is identical to the amino acid sequence of SEQ ID NO: 20.

56. The isolated antibody or antibody fragment of paragraph 55, wherein at least one framework region amino acid sequence of the heavy chain variable domain is at least 90% identical to at least one corresponding framework region amino acid sequence of a heavy chain variable domain amino acid sequence that is identical to the amino acid sequence of SEQ ID NO: 13.
57. The isolated antibody or antibody fragment of paragraph 55, wherein the isolated antibody or antibody fragment comprises a heavy chain variable domain amino acid sequence that is at least 90% identical to the amino acid sequence of SEQ ID NO: 13.
58. The isolated antibody or antibody fragment of paragraph 57, wherein the isolated antibody or antibody fragment comprises a heavy chain variable domain amino acid sequence that is identical to the amino acid sequence of SEQ ID NO: 13.
59. The isolated antibody or antibody fragment of paragraph 55, wherein at least one framework region amino acid sequence of the light chain variable domain is at least 90% identical to at least one corresponding framework region amino acid sequence of a light chain variable domain amino acid sequence that is identical to the amino acid sequence of SEQ ID NO: 17.
60. The isolated antibody or antibody fragment of paragraph 55, wherein the isolated antibody or antibody fragment comprises a light chain variable domain amino acid sequence that is at least 90% identical to the amino acid sequence of SEQ ID NO: 17.
61. The isolated antibody or antibody fragment of paragraph 60, wherein the isolated monoclonal antibody comprises a light chain variable domain amino acid sequence that is identical to the amino acid sequence of SEQ ID NO: 17.
62. The isolated antibody or antibody fragment of paragraph 555, wherein the isolated antibody or antibody fragment comprises a heavy chain variable domain amino acid sequence that is identical to the amino acid sequence of SEQ ID NO: 13 and a light chain variable domain amino acid sequence that is identical to the amino acid sequence of SEQ ID NO: 17.
63. An isolated antibody or antibody fragment comprising an amino acid sequence identical to SEQ ID NO: 12.
64. An isolated antibody or antibody fragment encoded by a nucleic acid comprising a nucleic acid identical to SEQ ID NO: 21.

The present disclosure is further illustrated by the following Example, which in no way should be construed as further limiting.

### EXAMPLES

### Example 1: scFV Anti-HPV Protein and Particle-Based Cellular Therapeutic Technology

This Example describes a method for targeted cancer therapy that comprises administering to a subject having a tumor (or tumor cells) a tumor-tropic virus-like particle (VLP) and an immune cell engineered to bind to the VLP.

Generation of experimental CAR constructs. Known antibody and nanobody sequences will be used to generate scFv antibodies that bind chicken egg ovalbumin (Reddy, 2010) and HPV L1 (Culp, 2007, Minaeian, 2012). Further antibody generation, e.g., for different virus proteins (e.g., a soluble pentamer (SP) derived from HPV, a virus-like particle (VLP) derived from HPV, or to a short peptide sequence (‘antigenic tag’) cloned into such an SP or VLP) will be performed using validated techniques (e.g., Ferrara, 2012) to derive fully human or humanized Vh and Vl sequences, scFv, F(ab′), F(ab)2 or full length antibody sequences from which can be readily derived the elements required for scFv construction suitable for CAR T cell use. For example, a scFv may encode a Variable Light Chain (Vl) sequence followed by a suitable linker (often a glycine/serine linker (e.g., G3S or G4S) which is linked to a Variable Heavy Chain (Vh) sequence, together cloned downstream of an appropriate leader sequence, e.g., a Vh or Vl leader sequence. Alternatively the Vh sequence is cloned upstream, followed by the Vl sequence. The scFv (Vl/Vh or Vh/Vl ) is then cloned in frame with a spacer sequence designed to keep the scFv above the cell surface, for this purpose IgGFc domain-derived and many other spacer sequences can be used. Other useful tag-binding sequences can be derived from heavy chain specific libraries (e.g., VHH) and from alternative scaffold libraries (e.g., Fibronectin Type III scaffold, ankyrin repeat protein, lipocalins). The transmembrane domain is usually derived from CD28, CD4, CD8 or other T cell membrane protein, fused in turn to the cytoplasmic domain. The cytoplasmic domain will contain CD3 zeta cytoplasmic sequences including the CD3 zeta signaling motif. The cytoplasmic domain will contain a costimulatory signaling motif encoded by one or more cytoplasmic domain sequences derived from CD28, ICOS, 4-1BB, CD27, OX-40, GITR, TNFRSF25, MYD88 or other signaling proteins.
Methods for encoding genes for CAR T cells. Various methods are available for the production of experimental CARs. For early POC in vitro studies, transient transfection of T cells with constructs encoding the CAR are often sufficient. For example, CAR constructs can be constructed to consist of a signal sequence, a scFv, an IgGFc-derived spacer, a transmembrane domain and the desired cytoplasmic domains. This CAR construct, or gene, is then cloned into a CMV promoter-based nonviral vector that are commercially available, e.g., the pmaxCloning vectors. For more robust studies, especially with regard to durable expression, the use of, and integration of, viral genomes is an advantage, as demonstrated by retrovirus, lentivirus, foamy virus and adeno-associated virus (AAV) vectors. Other useful systems include the transposable element sleeping beauty (Maiti et al. 2013. J. Immunother. 36: 112-123). Replication-deficient, lentiviral CAR vectors are produced as described in detail by Salmon and Trono (2007) who used Gibbon ape lentivirus to produce CAR genes for packaging (e.g., in 293T, Phoenix-eco, and other cell lines). Alternatively, a retroviral vector can be used to produce the pseudovirus particles. Vectors further optimized for gene expression include the MP 71 vector that has an optimized 5′ untranslated region (5′UTR). MP 71 is widely used for stable, high-level expression in T cells (Engels et al. 2003. Human Gene Therapy 14: 1155-1168). Multiple genes can be encoded in a single CAR expression construct by using, for example, in frame or independent IRES initiation sites for individual elements. Alternatively, inducible methods have been described, whereby the application of a small molecule can induce or block expression of one or more CAR elements. A wide variety of such methods are disclosed, such as inhibitory CARs, costimulatory CARs, “cideCARs”, on switches and others, the use of which can further modify or alter the activity of CAR T cells (see Baas, T. SciBX 7 (25); doi:10.1038/scibx.2014.725).
Generation of virus stocks encoding the cDNA for the experimental CAR. Cell lines capable of viral particle packaging are used to create high-titer viral stocks. For retroviral systems for example, the introduction of gag, env and pol genes along with the viral gene construct of interest is sufficient to allow efficient packaging. Most often, these genes are introduced using multiple plasmids, rendering the cells replication incompetent, as a safety measure. Lentiviral packaging systems are quite similar, and compatible with widely available permissive cell lines (e.g., modified Jurkat cell lines, the Lenti X 293T cell line (Clontech)). A recent development is the use of stable packaging cell lines e.g., 293FT (Sanber et al. 2015. Scientific Reports 5, article #9021). Culture supernatants are harvested, filtered, and concentrated by ultracentrifugation to collect pseudoviral particles that are then used to infect T cells. Virus can be aliquoted, flash-frozen in liquid nitrogen and stored at −80 C., preferably in liquid nitrogen.

Isolation, lentiviral transduction and expansion of human immune cells. As taught by Gallardo et al. (1997) and Brentjens et al. (2007), patient (tumor matched) or healthy peripheral blood mononuclear cells are isolated from whole blood or leukapheresis buffer by density centrifugation (e.g., Ficoll gradient). T cells are isolated using available and standardized techniques (e.g., cell sorting, magnetic bead separation, column separation by negative selection) and are incubated with agents that stimulate cell proliferation (e.g., anti-CD3/anti-CD28 beads, PMA/ionomycin). T cell cultures are diluted in media 1 day prior to use to stimulate transition into a cell division phenotype, and transduced by incubation with, for example, lentivirus particles, to introduce the viral genes to the cells. T cells are further cultured with anti-CD3/CD28-coated beads or with artificial antigen presenting cells, supplemented with IL-2, IL-7, IL-15, or other cytokines as needed, for one week prior to use. Flow cytometry, PCR or other standard methods are used to assess viral transduction efficiency by identifying CAR-expressing cells. To increase cell number further, activation periods can be alternated with resting periods of at least 3 days in IL-2 or other cytokines alone, prior to subsequent rounds of restimulation. Such methods allow multiple log expansion of cell number through additional rounds of rest and restimulation.

Papillomavirus virus-like particles (VLP) are made as described in Buck and Thompson (2007). Briefly, a large plasmid (>8 Kb) co-expressing the viral coat protein(s) (L1 and L2 or L1 alone) is transfected into 293TT cells. The viral coat protein(s) self-assemble into empty partially-assembled particles or protocapsids. After 48 hr, the cells are lysed, and then the partially-assembled particles or protocapsids are matured in vitro and purified by Optiprep density ultracentrifugation as described (Buck, 2007). Generation of modified VLPs can be done using three methodologies: 1) Addition of surface modifications (e.g., biotinylation, tumor antigens) can be made to the purified VLP through cell surface chemical linkages such as the lysine residues, followed by addition of the foreign antigen (e.g., Ovalbumin-streptavidin; Alexafluor-488-streptavidin) as described in Chackerian et al. (2008); 2) Addition of a succinimidyl ester group onto the foreign antigen such that direct interaction with the lysine residues of the VLP will allow for chemical linkage (e.g., Alexafluor 488-NHS ester; Roberts, 2007, see protocol at home.ccr.cancer.gov/lco/dyelabelinghpvcapsids.htm); 3) Insertion of a small epitope or tag within the L1 ORF to create chimeric VLPs. Insertion sites at aa140, aa179, aa266, aa283 and aa352 can successfully accommodate an insertion of up to 6aa and still allow for proper folding of L1 and VLP assembly and display the epitope on the surface of the VLP (Sadeyen, 2003). Additionally, amino acid sequences between 8-23aa can be inserted at aa440 or aa456 allowing for surface display of the epitope while maintaining VLP integrity (Matic, 2011). These chimeric VLPs can be produced and purified just as normal VLPs as previously described. Examples of antigenic tags include tag examples 6xHIS, Alexa488, FLAG, SV5 (simian virus 5 peptide) and many others of this class of small antigenic peptides, haptens and other molecules.

SP (also known as capsomers) can be generated using two different forms of L1 gene alterations: 1) deletion of the first nine amino acids of the N-terminus; the last 31 amino acids at the C-terminus; and helix 4 (Bishop, 2007); or 2) site-directed mutation of the cysteine at position 428 responsible for intermolecular disulfide bonds (Li, 1998; Sapp, 1998). For example, one can use a variant of the L1 428 mutant expression plasmid previously described in Buck et al. 2005 in which the cysteine at position 428 has been replaced with a serine residue (home.ccr.cancer.gov/Lco/pumL1B.txt). Production of the SP is similar to VLP production in the 293TT system however purification methods differ. Cell lysate is placed over a discontinuous Optiprep gradient 46%-30%-20%-15% (2.3 ml each) and centrifuged at 65000 rpm at 16° C. for 1 hr using an NVT65 rotor. Capsomers are localized to and collected from the middle of the gradient. Methods for production and purification of SP in E. coli are described in Bishop, 2007. Addition of antigenic tags is accomplished in the manner described for the VLPs.

Culture of target tumor cells with SP, VLP or tag-modified versions of SP or VLP. Incubation of tumor cells with SP, VLP or tag-modified versions of SP or VLP is done using published methods (see Kines et al. 2015. International journal of Cancer doi: 10.1002/ijc.29823). Briefly, tumor cells (tumor cell lines, patient-derived tumor cell lines, or patient derived primary tumor cell cultures) are dissociated and rotated on a nutating rocker at 37° C. for 2-4 hours in 10 ml of culture medium in order to recover their cell surface heparin sulfate proteoglycans (HSPG). SP, VLP or tag-modified versions of SP or VLP (2 ug/ml) are incubated with cells for 1 hr at 4° C. then washed gently with cold PBS containing 1% bovine serum albumin prior to further use. Heparin blockade of HSPG binding by viral particles or proteins serves as a specificity control. Immunodetection of surface bound SP, VLP or tag-modified versions of SP or VLP can then be assessed using fluorochrome conjugated antibodies directed against the VLP (or SP) as well as the modified epitope. Cells can then be analyzed by flow cytometry.
Co-culture of specific CAR T cells with targeted tumor cells to assess cytotoxicity. Cell lines representing diverse tumor cell types (e.g., lung, renal cell, ovarian, melanoma among many others), and/or cells isolated from resection/biopsy tumor tissue from patients, will be incubated with purified viral proteins and particles of the present disclosure. Tumor cells are seeded in a 96-well plate at various concentrations e.g., between 1×10⁴ cells/well and 1×10⁶ cells/well and incubated with SP, VLP or tag-modified versions of SP or VLP in the presence or absence of heparin to block HSPG interaction. After one hour, cells are washed and CART cells are added at different effector:target ratios (e.g., (1:1, 1:5, 5:1, 10:1, 1:10 etc). After incubation at 37° C. for 24-48 hours, cytotoxicity is determined by using standard techniques, e.g., an LDH release assay or other methods that measure cell membrane integrity. The mechanism of action of cytotoxicity is well understood and involves the release of granzyme B and perforin from the T cells, usually accompanied by the production of IFN gamma. These secreted products are readily measured by ELISA, multiplex, bead array and other standard assays. In addition, supernatant and T cells are collected. Supernatant is analyzed for the presence of T cell cytokines including interferon gamma and IL-2. T cells are assessed for their activation state or proliferative state using flow cytometry techniques including cell surface marker staining and CSFE staining.
In vivo validation of SP, VLP or tag-modified versions of SP or VLP, targeting tumor cells. Syngeneic or xenograft tumors are established in matched mouse strains or immunodeficient (e.g., NOD/SCID) mice and are allowed to grow until palpable tumors are obtained. Mice with tumors of 50-100 mm³ are randomized for treatment. Dye-labeled forms of SP, VLP or tag-modified versions of SP or VLP (e.g., 10-500 ug) are then administered by IV or IP injection. Two hours after injection of SP, VLP or tag-modified versions of SP or VLP, tumors are excised and examined for viral particle/protein binding by analysis of specific dyes (see for example Kines et al. 2015. International journal of Cancer doi: 10.1002/ijc.29823) or by staining cells with anti-VLP or SP antibodies. The optimal concentration of SP, VLP or tag-modified versions of SP or VLP for each tested tumor type will be used in the in vivo protocol.
In vivo cytotoxicity of CAR T cells directed to tumor cells coated with SP, VLP or tag-modified versions of SP or VLP. Syngeneic or xenograft tumors are established in matched mouse strains or immunodeficient (e.g., NOD/SCID) mice and are allowed to grow until palpable tumors are obtained. Xenograft tumors will include human tumor cell lines, patient derived (PDX) cell lines and/or primary human patient tumors. Mice with tumors of 50-100 mm³ are randomized for treatment. The optimal concentration of SP, VLP or tag-modified versions of SP or VLP is administered IV, IP or intratumorally (IT). Two hours later, CAR T-cells (10⁵-10⁹) are injected via various routes, preferably IV, IP or IT. Tumor volumes and mouse survival are measured. In additional experiments, tumor volume is allowed to exceed 100 mm³ (e.g., 200-500 mm³) in order to measure tumor regression in the presence of CAR T cell therapy. An example of a tumor model is a xenograft model of ovarian cancer. For an established ovarian cancer model, 6- to 12-week-old female NNOD/SCID or NOD/SCID/common gamma chain deficient mice are inoculated subcutaneously with 1×10⁶ A1847, SKOV3 or OVCAR (e.g., lines 2, 3, or 5) cells on the flank on day 0. After tumors become palpable at about 6 weeks, human primary T cells are activated, and transduced as described. After 2 weeks of T cell expansion, the tumor burden will be >100 mm³. The optimal concentration of SP, VLP or tag-modified versions of SP or VLP is administered IV, IP or intratumorally (IT). Two hours later, mice are then injected IV with CAR T cells. For the intraperitoneal model of ovarian cancer, 6 to 12-week-old NOD/SCID or NOD/SCID/common gamma chain deficient mice are injected IP with 10×10⁶ A1847, SKOV3 or OVCAR (e.g., lines 2, 3, or 5) cells. Two weeks after peritoneal inoculation, mice bearing established A1847 tumors are given the optimal concentration of SP, VLP or tag-modified versions of SP or VLP administered IV, IP or intratumorally (IT), followed two hours later by CAR T cells IV. Mice are sacrificed when they became distressed or moribund and the tumor mass is quantified, preferentially by imaging (e.g., of luciferase-expressing tumor cells). In all experiments, blood is collected throughout the duration to measure CAR T cell proliferation and expansion, and to assess cytokine secretion. Subsets of the animals are humanely euthanized at various time points to measure tumor infiltration and tumor viability.

### Example 2: Construction and Use of the SP-CAR

Soluble protein (SP; also known as capsomers) can be encoded using two different forms of L1 gene alterations: 1) deletion of the first nine amino acids of the N-terminus, the last 31 amino acids at the C-terminus, and helix 4 (Bishop, 2007); or 2) site-directed mutation of the cysteine at position 428 responsible for intermolecular disulfide bonds (Li, 1998; Sapp, 1998). For example, one can use a variant of the L1 428 mutant expression plasmid previously described in Buck et al. 2005 in which the cysteine at position 428 has been replaced with a serine residue.

These SP mutants can be encoded as elements of CAR constructs using several different strategies. In one example, a single mutated L1 gene is encoded in frame as the antigen-binding domain of a CAR gene, e.g., mutated L1 followed by an IgGFc-derived spacer, a transmembrane domain and the desired cytoplasmic domains. Such a construct would require independent or bicistronic expression of mutated L1 protein. In this scenario, some of the expressed protein will assemble with the CAR-encoded mutant L1 protein to form pentamers (the preferred form that the expressed L1 mutated proteins will take). In another example the L1 mutated genes are encoded with five copies in frame as described above, separated with linkers to allow pentameric folding. An example of a flexible linker is G4S, although the precise size and geometry needed to optimize expression and pentameric formation is subject to discovery.

The CAR construct, or gene, is then cloned into CMV promoter-based nonviral vectors that are commercially available, e.g., the pmaxCloning vectors. For more robust studies, especially with regard to durable expression, the use of, and integration of, viral genomes is an advantage, as demonstrated by retrovirus, lentivirus, foamy virus and adeno-associated virus (AAV) vectors. Other useful systems include the transposable element sleeping beauty (Maiti et al. 2013. J. Immunother. 36: 112-123). Replication-deficient, lentiviral CAR vectors are produced as described in detail by Salmon and Trono (2007) who used Gibbon ape lentivirus to produce CAR genes for packaging (e.g., in 293T, Phoenix-eco, and other cell lines). Alternatively, a retroviral vector can be used to produce the pseudovirus particles. Vectors further optimized for gene expression include the MP 71 vector that has an optimized 5′ untranslated region (5′UTR). MP 71 is widely used for stable, high-level expression in T cells (Engels et al. 2003. Human Gene Therapy 14: 1155-1168). Multiple genes can be encoded in a single CAR expression construct by using, for example, in frame or independent IRES initiation sites for individual elements. Alternatively, inducible methods have been described, whereby the application of a small molecule can induce or block expression of one or more CAR elements. A wide variety of such methods are disclosed, such as inhibitory CARs, costimulatory CARs, “cideCARs”, on switches and others, the use of which can further modify or alter the activity of CAR T cells (see Baas, T. SciBX 7 (25); doi:10.1038/scibx.2014.725).

### Example 3: Generation of scFv Antibody Fragments That Bind to Human Papillomavirus (HPV) Particles

Yeast display naïve human antibody library, antibodies, biotinylation kit, cells. A large yeast display naïve single chain variable fragment (scFv) human antibody library was constructed using a collection of human antibody gene repertoires, including the genes used for the construction of a phage display Fab library (Zhu Z et al., 2009, Methods Mol Biol 525, 129-142).

Mouse monoclonal anti-c-Myc antibody was purchased from Roche (Pleasanton, Calif.). PE-conjugated streptavidin and Alexa-488 conjugated goat anti-mouse antibody were purchased from Invitrogen (Carlsbad, Calif.). Yeast plasmid extraction kits were purchased from Zymo Research (Irvine, Calif.). 293 free-style protein expression kits were purchased from Invitrogen. An AutoMACS System was purchased from Miltenyi Biotec (Cologne, Germany).

Yeast display human antibody library sorting on AutoMACS. Biotinylated HPVL1 virus like particle (VLP) was used as the target for three rounds of sorting of the initial yeast display naïve human antibody library. Approximately 5×10¹⁰ cells from the initial naïve antibody library and 10 μg of biotinylated HPVL1 VLP were incubated in 50 ml PBSA (phosphate-buffered saline containing 0.1% bovine serum albumin) at room temperature (RT) for 2 hr with rotation. The mixture of biotinylated HPVL1 VLP bound to displayed antibody on yeast cells from the library was washed three times with PBSA and incubated with 100 μl of streptavidin conjugated microbeads at RT from Miltenyi Biotec. The resultant mixture was washed once with PBSA and loaded onto the AutoMACS system for the first round of sorting. The sorted cells were amplified in SDCAA media (20 g dextrose, 6.7 g Difco yeast nitrogen base w/o amino acids, 5 g Bacto casamino acids, 5.4 g Na₂HPO₄ and 8.56 g NaH₂PO₄. H₂O in 1 liter water) at 30° C. and 250 rpm for 24 hr. The culture was then induced in SGCAA media (20 g galactose, 20 g raffinose , 1 g dextrose, 6.7 g Difco yeast nitrogen base w/o amino acids, 5 g Bacto casamino acids, 5.4 g Na₂HPO₄ and 8.56 g NaH₂PO₄. H₂O in 1 liter water) at 20 ° C. and 250 rpm for 16-18 hr. Then the amplified pool was used for the next round of sorting.

Cloning, Expression and purification of scFv proteins. Plasmids were extracted from the enriched yeast pool after three rounds of sorting using yeast plasmid extraction kits (Zymo Research), following the manufacturer's instructions. Extracted plasmids were transformed into 10G chemical competent E. coli (Lucigen , Middleton, Wis.) for further amplification; The scFv-encoding inserts of the pool were digested with SfiI and ligated into modified pIgD bearing the same set of SfiI sites for soluble expression. plasmids extracted from the random clones derived from the scFv-Fc cloning were sent for DNA sequencing to obtain the nucleic acid sequences encoding the positive binder antibodies. These constructs were transformed into HB2151 cells for expression and the scFvs were purified using Ni-NTA agarose following the manufacturer's (Qiagen) protocol.

ELISA binding assay. 50 μl of the diluted HPV or BPV in PBS at 2 ug/ml was coated in a 96-well plate at 4° C. overnight. Purified scFv with His and Flag tags was serially diluted and added into the target protein coated wells. After washing, a 1:3000 diluted HRP conjugated anti-Flag antibody was added for 1 hr at RT. After washing, 3,3,5,5′-Tetramethylbenzidine (TMB) substrate was added, 1N H₂SO₄ was added to stop the reaction after incubation at room temperature for 10 minutes and the O.D. was read at 450 nm.

### Example 4: scFv Antibody Fragments Bind to Human Papillomavirus (HPV) Particles Bound to the Surface of Tumor Cells In Vitro

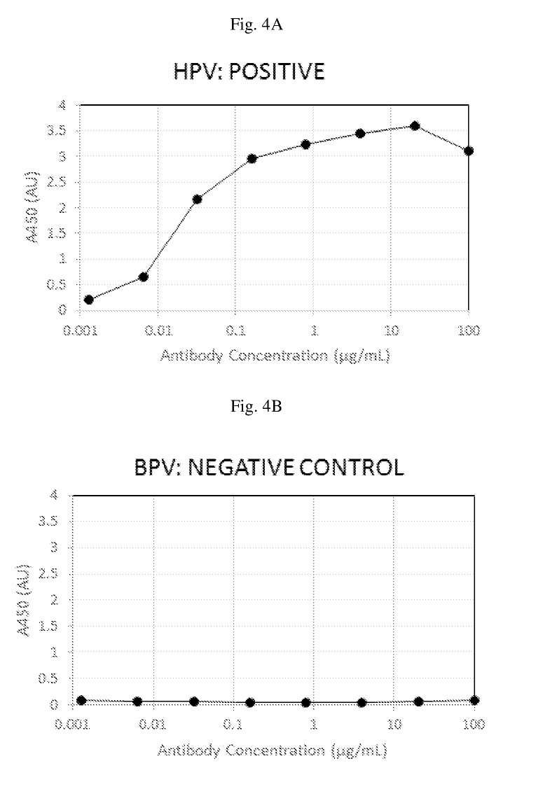

FIG. 4 shows data demonstrating that the scFv antibody fragments bind to human papillomavirus (HPV) particles bound to the surface of tumor cells in vitro.

HeLa cells were harvested with 10 mM EDTA and allowed to recover in a suspension of DMEM+10% FBS for 4 hr on a nutating rocker at 37° C., 5% CO₂ to allow for surface HSPG recovery. 1×10⁵ cells were added to wells of a 96-well U-bottom culture plate and incubated with PBS+2% FBS (flow buffer; used for all washes), 10 μg/ml of HPV16 L1 VLPs or 10 μg/ml of HPV16 L1/L2 VLPs were added for 1 hr in 100 μl at 4° C. Cells were washed once with flow buffer then blocked with 100 μl of 10% donkey serum diluted in flow buffer for 30 min at 4° C. Cells were washed once then incubated with buffer alone or 1 μg/ml of each of the provided scFv clones (#1 and #2; tagged with DYKDDDDK tag (SEQ ID NO: 1)) for 1 hr in 100 μl at 4° C. Cells were washed twice with flow buffer then incubated with rat anti-DYKDDDDK tag (SEQ ID NO: 1) (clone L5, Biolegend #637301) in 100 μl at 5 μg/ml for 1 hr at 4° C. Cells were washed twice with flow buffer then incubated with donkey anti-rat*Alexafluor 488 (Jackson ImmunoResearch Inc #712-545-153) at 1 μg/ml in 100 μl for 1 hr at 4° C. Cells were washed twice with flow buffer followed by 10 min fixation with 4% paraformaldehyde and washed once with flow buffer. Cells were acquired on a BD FACS Canto II, healthy cells were gated from the forward and side scatter plots. Data shown are histograms representing the alexafluor 488 populations. Controls included unstained cells, cells stained with scFv and all antibodies but without VLPs, cells bound by VLPs and stained with a rat anti-sera raised against HPV16 (NIH).

### Example 5: Immune Cells Expressing scFV-L1 Capsomer Target Surface HSPG on Tumor Cells

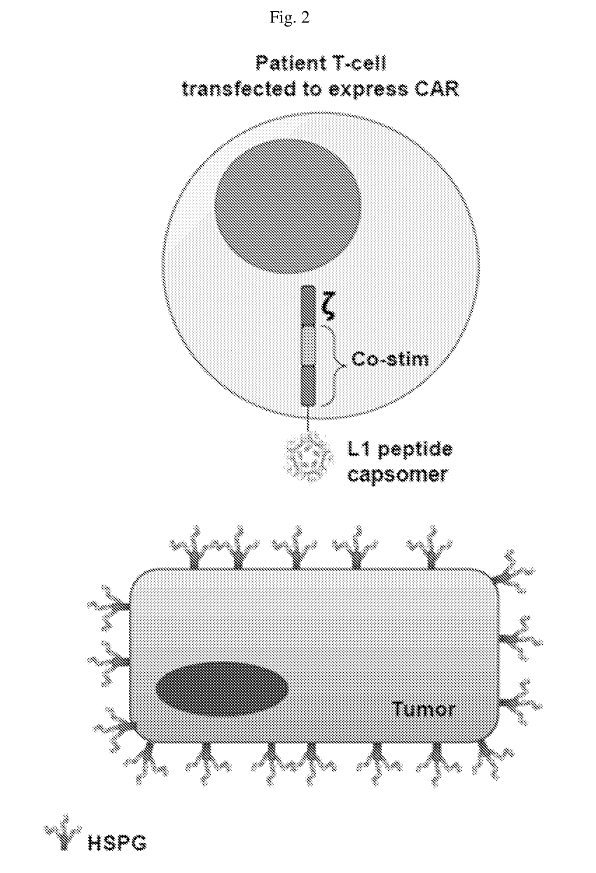

In this example, an immune cell (e.g., a T cell) is engineered to express a chimeric antigen receptor (CAR) that comprises a single chain antibody fragment (scFv) that comprises a tumor-tropic L1 peptide capsomer. The L1 peptide capsomer homes to/binds to HSPGs located on the surface of a tumor/tumor cells. Thus, the engineered immune cell, expressing the scFV, is capable of homing to the tumor cell(s) (FIG. 2).

CAR expression lentiviral vectors are designed as described in Example 1. Surface expression of PV capsomers on immune cells (e.g., T cells) is confirmed using standard immuno-imaging approaches, or as described in Example 1.

In vitro confirmation of binding between immune cells (e.g., T cells) transfected with the capsomer vector and tumor cells is performed as described above, or using standard techniques known in the art (e.g., immunostaining or electron microscopy after co-incubation of T cells and tumor cells).

In vivo data is obtained using placenta-derived multipotent cells (PDMCs) transduced with lentiviral CAR in NOD-SCID/gamma^{-/-} mice with human tumors (e.g., ovarian tumors).

### Example 6: Immune Cells Expressing scFv Anti-HSPG Protein Target Surface HSPG on Tumor Cells

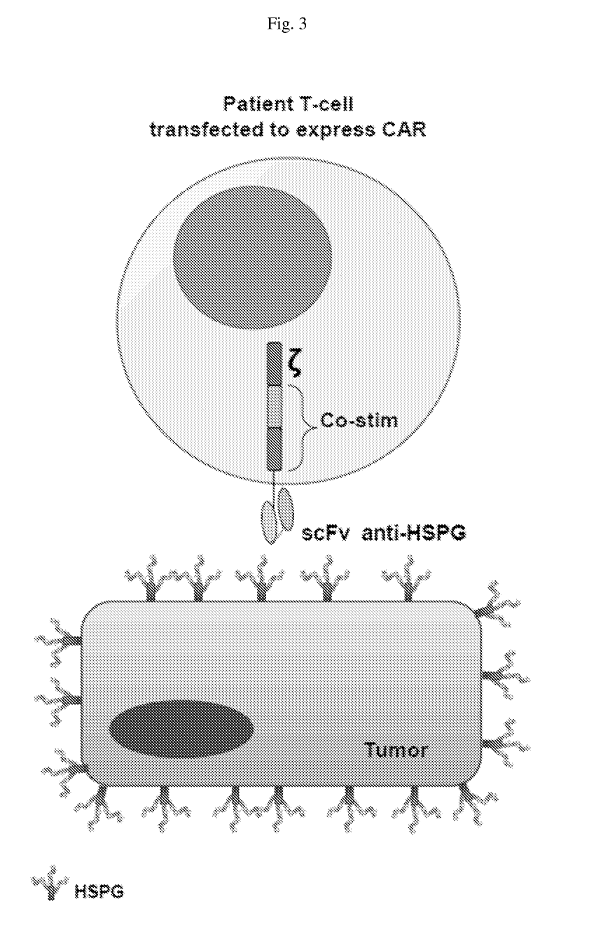

In this example, an immune cell (e.g., a T cell) is engineered to express a chimeric antigen receptor (CAR) that comprises a scFv that comprises an anti-HSPG receptor. The anti-HSPG receptor guides the immune cell to HSPGs located on the surface of a tumor/tumor cells. Thus, the engineered immune cell, expressing the scFv, is capable of homing to the tumor cell(s) (FIG. 3).

scFv against PV are generated using phage display libraries. Phage library panning against both VLPs and capsomers are performed to generate a dual-use scFv.

CAR expression lentiviral vectors are designed as described in Example 1.

Capsomer binding specificity and feasibility is performed both in vitro and in vivo as described in Example 1.

In vitro data is obtained using T cells transfected with the CAR expression vector in combination with tumor cells bound by VLPs and capsomers.

In vivo data is obtained using human PDMCs transduced with lentiviral CAR in NOD-SCID/gamma^{-/-} mice with human tumors (e.g., ovarian tumors).

scFv Sequences

HPV scFv #1
VH-Linker-VL
QVQLQQSGPGLVKPSQTLSLTCAISGDSVSSNSAAWNWIRQSPSRGLEWL
GRTYYRSKWYNDYAVSVKSRITIDPDTSKNQFSLQLNSVTPEDTAMYYCA
REGDDAFDIWGQGTMVTVSSGGGGSGSGASSGGGSSYVLTQLPSVSVSPG
QTARITCGGNNIGSKSVHWYQQKPGQAPVLVVYDDSDRPSGIPERFSGS
NSGNTATLTISGTQAMDEADYYCQAWDSGTAVFGGGTKVTVLG
(SEQ ID NO: 2)
*3HCDRs underlined and 3LCDRs double underlined
(IMGT definition), Linker labeled in purple
HPV scFV #1 Heavy Chain
QVQLQQSGPGLVKPSQTLSLTCAISGDSVSSNSAAWNWIRQSPSRGLEWL
GRTYYRSKWYNDYAVSVKSRITIDPDTSKNQFSLQLNSVTPEDTAMYYCA
REGDDAFDIWGQGTMVTVSS (SEQ ID NO: 3)
HCDR1
GDSVSSNSAA (SEQ ID NO: 4)
HCDR2
TYYRSKWYN (SEQ ID NO: 5)
HCDR3
AREGDDAFD (SEQ ID NO: 6)
HPV scFV #1 Light Chain
SYVLTQLPSVSVSPGQTARITCGGNNIGSKSVHWYQQKPGQAPVLVVY
DDSDRPSGIPERFSGSNSGNTATLTISGTQAMDEADYYCQAWDSGTAVF
GGGTKVTVLG (SEQ ID NO: 7)
LCDR1
NIGSKS (SEQ ID NO: 8)
LCDR2
DDS (SEQ ID NO: 9)
LCDR3
QAWDSGTAV (SEQ ID NO: 10)
HPV scFv #1 Nucleotide Sequence
CAGGTACAGCTGCAGCAGTCAGGTCCAGGACTGGTGAAGCCCTCGCAGAC
CCTCTCACTCACCTGTGCCATCTCCGGGGACAGTGTCTCTAGCAACAGTG
CTGCTTGGAACTGGATCAGGCAGTCCCCATCGAGAGGCCTTGAGTGGCTG
GGAAGGACATACTACAGGTCCAAGTGGTATAATGATTATGCAGTATCTGT
GAAAAGTCGAATAACCATCGACCCAGACACATCCAAGAACCAGTTCTCCC
TGCAGCTGAACTCTGTGACTCCCGAGGATACGGCTATGTATTACTGTGCA
AGAGAGGGAGATGATGCTTTTGATATCTGGGGCCAAGGGACAATGGTCAC
CGTCTCCTCAGGAGGTGGCGGGTCTGGTAGTGGCGCTAGCAGTGGTGGCG
GATCCTCCTATGTGCTGACTCAGCTACCCTCGGTGTCAGTGTCCCCAGGA
CAGACGGCCAGGATTACCTGTGGGGGAAACAACATTGGAAGTAAAAGTGT
GCACTGGTACCAGCAGAAGCCAGGCCAGGCCCCTGTGCTGGTCGTCTATG
ATGATAGCGACCGGCCCTCAGGGATCCCTGAGCGATTCTCTGGCTCCAAC
TCTGGGAACACAGCCACTCTGACCATCAGCGGGACCCAGGCTATGGATGA
GGCTGACTATTACTGTCAGGCGTGGGACAGCGGCACTGCGGTATTCGGCG
GAGGGACCAAGGTCACCGTCCTAGGT (SEQ ID NO: 11)
HPV scFv #2
VH-Linker-VL
QVQLQQSGPGLVKPSQTLSLTCAISGDSVSSYNAAWTWIRQSPSRGLEWL
GRTYYGSKWYTDYAVSVKSRITINPDTSKNQFSLQLNSVTPEDTAVYYCA
RESELRADAFDIWGQGTTVTVSSGGGGSGSGASGGGGSSSELTQDPAVSV
ALGQTVRITCQGDSLRSYYASWYQQKPGQAPVLVIYGKNNRPSGIPDRF
SGSSSGTTASLTITGAQAEDEADYYCNSRDSSGNHLVFGGGTKVTVLG
(SEQ ID NO: 12)
*3HCDRs underlined and 3LCDRs double underlined
(IMGT definition), Linker labeled in purple
HPV scFV #2 Heavy Chain
QVQLQQSGPGLVKPSQTLSLTCAISGDSVSSYNAAWTWIRQSPSRGLEWL
GRTYYGSKWYTDYAVSVKSRITINPDTSKNQFSLQLNSVTPEDTAVYYCA
RESELRADAFDIWGQGTTVTVSS (SEQ ID NO: 13)
HCDR1
GDSVSSYNAA (SEQ ID NO: 14)
HCDR2
TYYGSKWYT (SEQ ID NO: 15)
HCDR3
ARESELRADAFDI (SEQ ID NO: 16)
HPV scFV #2 Light Chain
SSELTQDPAVSVALGQTVRITCQGDSLRSYYASWYQQKPGQAPVLVIY
GKNNRPSGIPDRFSGSSSGTTASLTITGAQAEDEADYYCNSRDSSGNH
LVFGGGTKVTVLG (SEQ ID NO: 17)
LCDR1
SLRSYY (SEQ ID NO: 18)
LCDR2
GKN (SEQ ID NO: 19)
LCDR3
NSRDSSGNHLV (SEQ ID NO: 20)
HPV scFv #2 Nucleotide Sequence
CAGGTACAGCTGCAGCAGTCAGGTCCAGGACTGGTGAAGCCCTCGCAGAC
CCTCTCACTCACCTGTGCCATCTCCGGGGACAGTGTCTCTAGCTACAATG
CTGCTTGGACCTGGATCAGGCAGTCCCCATCGAGAGGCCTTGAGTGGCTG
GGAAGGACATACTACGGGTCCAAGTGGTATACTGATTATGCAGTATCTGT
GAAAAGTCGAATAACCATCAACCCAGACACATCCAAGAACCAGTTCTCCC
TGCAGCTGAACTCTGTGACTCCCGAGGACACGGCTGTGTATTACTGTGCA
AGAGAATCAGAGCTACGGGCTGATGCTTTTGATATCTGGGGCCAAGGGAC
CACGGTCACCGTCTCCTCAGGAGGTGGCGGGTCTGGTAGTGGCGCTAGCG
GTGGTGGCGGATCCTCTTCTGAGCTGACTCAGGACCCTGCTGTGTCTGTG
GCCTTGGGACAGACAGTCAGGATCACATGCCAAGGAGACAGCCTCAGAAG
CTATTATGCAAGCTGGTACCAGCAGAAGCCAGGACAGGCCCCTGTACTTG
TCATCTATGGTAAAAACAACCGGCCCTCAGGGATCCCAGACCGATTCTCT
GGCTCCAGCTCAGGAACCACAGCTTCCTTGACCATCACTGGGGCTCAGGC
GGAAGATGAGGCTGACTATTACTGTAACTCCCGGGACAGCAGTGGTAACC
ATCTGGTATTCGGCGGAGGGACCAAGGTCACCGTCCTAGGT
(SEQ ID NO: 21)

### REFERENCES

Brentjens R J, Santos E, Nikhamin Y, Yeh R, Matsushita M, La Perle K, Quintás-Cardama A, Larson S M, Sadelain M. Genetically targeted T cells eradicate systemic acute lymphoblastic leukemia xenografts. Clin Cancer Res. 2007 Sep. 15; 13 (18 Pt 1):5426-35.
Bishop B, Dasgupta J, Klein M, Garcea R L, Christensen N D, Zhao R, Chen X S. Crystal structures of four types of human papillomavirus L1 capsid proteins: understanding the specificity of neutralizing monoclonal antibodies. J Biol Chem. 2007 Oct. 26; 282 (43):31803-11.
Buck C B, Thompson C D, Pang Y Y, Lowy D R, Schiller J T. Maturation of papillomavirus capsids. J Virol. 2005 March; 79 (5):2839-46.
Chackerian B, Durfee M R, Schiller J T. Virus-like display of a neo-self antigen reverses B cell anergy in a B cell receptor transgenic mouse model. J Immunol. 2008 May 1; 180 (9):5816-25.
Culp T D, Spatz C M, Reed C A, Christensen N D. Binding and neutralization efficiencies of monoclonal antibodies, Fab fragments, and scFv specific for L1 epitopes on the capsid of infectious HPV particles. Virology. 2007 May 10; 361 (2):435-46.
Davila M L, Kloss C C, Gunset G, Sadelain M. CD19 CAR-targeted T cells induce long-term remission and B Cell Aplasia in an immunocompetent mouse model of B cell acute lymphoblastic leukemia. PLoS One. 2013 Apr. 9; 8 (4):e61338.
Ferrara F, et al., Using phage and yeast display to select hundreds of monoclonal antibodies: application to Antigen 85, a tuberculosis biomarker. PLoS One 2012 7 (11): e49535
Gallardo H F, Tan C, Ory D, Sadelain M. Recombinant retroviruses pseudotyped with the vesicular stomatitis virus G glycoprotein mediate both stable gene transfer and pseudotransduction in human peripheral blood lymphocytes. Blood. 1997 Aug. 1; 90 (3):952-7.
Lee J, Sadelain M, Brentjens R. Retroviral transduction of murine primary T lymphocytes. Methods Mol Biol. 2009; 506:83-96.
Li M, Beard P, Estes P A, Lyon M K, Garcea R L. Intercapsomeric disulfide bonds in papillomavirus assembly and disassembly. J Virol. 1998 March; 72 (3):2160-7.
Matić S, Rinaldi R, Masenga V, Noris E. Efficient production of chimeric human papillomavirus 16 L1 protein bearing the M2e influenza epitope in Nicotiana benthamiana plants. BMC Biotechnol. 2011 Nov. 15; 11:106. doi: 10.1186/1472-6750-11-106.
Minaeian S, Rahbarizadeh F, Zarkesh-Esfahani S H, Ahmadvand D, Broom O J. Neutralization of human papillomavirus by specific nanobodies against major capsid protein Ll. J Microbiol Biotechnol. 2012 May; 22 (5):721-8.
Reddy S T, Ge X, Miklos A E, Hughes R A, Kang S H, Hoi K H, Chrysostomou C, Hunicke-Smith S P, Iverson B L, Tucker P W, Ellington A D, Georgiou G. Monoclonal antibodies isolated without screening by analyzing the variable-gene repertoire of plasma cells. Nat Biotechnol. 2010 September; 28 (9):965-9.
Roberts J N, Buck C B, Thompson C D, Kines R, Bernardo M, Choyke P L, Lowy D R, Schiller J T. Genital transmission of HPV in a mouse model is potentiated by nonoxynol-9 and inhibited by carrageenan. Nat Med. 2007 July; 13 (7):857-61.
Sadeyen J R, Tourne S, Shkreli M, Sizaret P Y, Coursaget P. Insertion of a foreign sequence on capsid surface loops of human papillomavirus type 16 virus-like particles reduces their capacity to induce neutralizing antibodies and delineates a conformational neutralizing epitope. Virology. 2003 Apr. 25; 309 (1):32-40.
Salmon P, Trono D. Production and titration of lentiviral vectors. Curr Protoc Hum Genet. 2007 July; Chapter 12: Unit 12.10.
Sapp M, Fligge C, Petzak I, Harris J R, Streeck R E. Papillomavirus assembly requires trimerization of the major capsid protein by disulfides between two highly conserved cysteines. J Virol. 1998 July; 72 (7):6186-9.

### Equivalents

Those skilled in the art will recognize, or be able to ascertain using no more than routine experimentation, many equivalents to the specific embodiments of the disclosure described herein. Such equivalents are intended to be encompassed by the following claims.

All references, including patent documents, disclosed herein are incorporated by reference in their entirety.

What is claimed is:

1. A method comprising
delivering to a subject a papillomavirus particle or soluble papillomavirus protein that targets a tumor, and
delivering to the subject an immune cell expressing a receptor that binds to a surface antigen of the papillomavirus particle or soluble papillomavirus protein, respectively.

2. The method of claim 1, wherein the papillomavirus particle is a papilloma virus-like particle or a papilloma pseudovirus.

3. The method of claim 1, wherein the papillomavirus particle or soluble papillomavirus protein is a human papillomavirus particle or a soluble human papillomavirus protein.

4. The method of claim 1, wherein the papillomavirus particle or soluble papillomavirus protein is a modified human papillomavirus particle or a soluble modified human papillomavirus protein.

5. The method of claim 1, wherein the papillomavirus particle or soluble papillomavirus protein is a non-human papillomavirus particle or a soluble non-human papillomavirus protein.

6. The method of claim 4, wherein the non-human papillomavirus particle is a bovine, murine, cotton-rabbit, macaque or rhesus papillomavirus particle or soluble papillomavirus protein.

7. The method of claim 6, wherein the non-human papillomavirus particle or soluble non-human papillomavirus protein is a bovine papillomavirus particle or soluble bovine papillomavirus protein.

8. The method of claim 1, wherein the papillomavirus particle is a papilloma virus-like particle.

9. The method of claim 1, wherein the papillomavirus particle is a papilloma pseudovirus.

10. The method of claim 1, wherein the soluble papillomavirus protein forms a capsomer.

11. The method of claim 1, wherein the capsomer comprises of L1 protein.

12. The method of claim 1, wherein the immune cell is leukocyte.

13. The method of claim 12, wherein the leukocyte is a neutrophil, eosinophil, basophil, lymphocyte or a monocyte.

14. The method of claim 13, wherein the leukocyte is a lymphocyte.

15. The method of claim 14, wherein the lymphocyte is a T cell, a B cell, an NK cell, or an NKT cell.

16. The method of claim 15, wherein the lymphocyte is a T cell.

17. The method of claim 1, wherein immune cell is a dendritic cell.

18. The method of claim 1, wherein the receptor is a recombinant antigen receptor.

19. The method of claim 1, wherein the receptor is a chimeric antigen receptor.

20. The method of claim 1, wherein the surface antigen of the papillomavirus particle is an L1 protein or an L1/L2 protein complex.

21. The method of claim 1, wherein the surface antigen is covalently linked to a surface of the papillomavirus particle.

22. The method of claim 21, wherein the surface antigen is a hapten.

23. The method of claim 1, wherein the surface antigen is non-covalently linked to a surface of the papillomavirus particle.

24. The method of claim 1, wherein the surface antigen is a peptide incorporated into a region of a recombinant capsid protein that is surface-exposed in the papillomavirus particle.

25. The method of claim 1, wherein the surface antigen of the papillomavirus is a self-antigen.

26. The method of claim 1, wherein the surface antigen of the papillomavirus is a non-self antigen.

27. The method of claim 26, wherein the non-self antigen is a bacterial, yeast, protozoan or viral antigen.

28. The method of claim 1, wherein the surface antigen of the papillomavirus is a tumor antigen.

29. The method of claim 28, wherein the tumor antigen is a tumor-specific antigen (TSA) or a tumor-associated antigen (TAA).

30. The method of claim 29, wherein the tumor antigen is or comprises an epitope of CD19, CD20, CD21, CD22, CD45, BCMA, MART-1, MAGE-A3, glycoprotein 100 (gp100), NY-ESO-1, HER2 (ErbB2), IGF2B3, EGFRvIII, Kallikrein 4, KIF20A, Lengsin, Meloe, MUC-1, MUC5AC, MUC-16, B7-H3, B7-H6, CD70, CEA, CSPG4, EphA2, EpCAM, EGFR family, FAP, FRα, glupican-3, GD2, GD3, HLA-A1+MAGE1, IL-11Rα, IL-23Rα2, Lewis-Y, mesothelin, NKG2D ligands, PSMA, ROR1, survivin, TAG72 or VEGFR2.

31. The method of claim 30, wherein the tumor antigen is or comprises an epitope of CD19.

32. The method of claim 30, wherein the tumor antigen is selected from full length CD19, a fragment of CD19, at least one C2 Ig-like domain of CD19, or a linear epitope of CD19.

33. The method of claim 1, wherein the surface antigen of the papillomavirus is or comprises a synthetic epitope.

34. The method of claim 33, wherein the synthetic epitope is a His tag, a FLAG tag, or an SV5 tag.

35. The method of claim 1, wherein the papillomavirus particle, the immune cell or the papillomavirus particle and the immune cell are delivered via a parenteral, enteric or topical route.

36. The method of claim 35, wherein the parenteral route is intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumor, intratympanic, intrauterine, intravascular, intravenous (bolus or drip), intraventricular, intravesical or subcutaneous.

37. The method of claim 1, wherein the tumor is an ocular tumor, a melanoma, a head and neck tumor, a lung tumor, a bladder tumor, a breast tumor, a colorectal tumor, a gastric tumor, an ovarian tumor, a pancreatic tumor, a prostate tumor, a liver tumor, a renal tumor, or mesothelioma].

38. A method comprising
delivering to a subject a virus that targets a tumor, and
delivering to the subject an immune cell comprising a receptor that binds to a surface antigen of the virus.
